(19) United States
(12) Patent Application Publication
(10) Pub. No.: US 2017/0027955 A1
(43) Pub. Date: Feb. 2, 2017

(54) EXPRESSION LEVELS OF BCL-XL, BCL2, BCL-W, AND BAD AND CANCER THERAPIES

(71) Applicant: Constellation Pharmaceuticals, Inc., Cambridge, MA (US)

(72) Inventors: Andrew Ryan Conery, Belmont, MA (US); Jennifer Andrea Mertz, Medford, MA (US); Robert Joseph Sims, III, Dracut, MA (US); Richard C. Centore, Medford, MA (US)

(21) Appl. No.: 15/301,770

(22) PCT Filed: Apr. 2, 2015
(86) PCT No.: PCT/US2015/024058

§ 371 Date: Oct. 4, 2016

Related U.S. Application Data
(60) Provisional application No. 61/975,432, filed on Apr. 4, 2014.

(51) Int. Cl. A61K 31/55

(52) U.S. Cl. CPC A61K 31/55

(57) ABSTRACT

Described herein are novel methods for treating subjects with a cancer which is not overexpressing at least one of BCL-xL, BCL-w, and BAD, and optionally further overexpressing BCL-2. Also provided herein are tools for determining and/or assessing, and for the administration of, cancer treatments involving BET bromodomain inhibitors, BCL-xL inhibitors, or combinations thereof.

### RELATED APPLICATIONS

This application claims priority to U.S. Provisional Application No. 61/975,432, filed Apr. 4, 2014. The entire contents of the above mentioned application are incorporated herein by reference.

### TECHNICAL FIELD OF THE INVENTION

Provided herein are methods of treating subjects with a cancer which is expressing BCL-xL. Also provided herein are tools for determining and/or assessing, and for the administration of, cancer treatments involving BET bromodomain inhibitors, BCL-xL inhibitors, or combinations thereof.

Also provided herein are methods of treating subjects with a cancer which is expressing BCL-2, BCL-w, or Bcl-2-associated death promoter (BAD). Also provided herein are tools for determining and/or assessing, and for the administration of, cancer treatments involving BET bromodomain inhibitors, BCL-w inhibitors, BAD inhibitors, or combinations thereof.

Further provided are methods of treating subjects with a cancer which is expressing two or more of BCL-xL, BCL-2, BCL-w, and Bcl-2-associated death promoter (BAD). Also provided herein are tools for determining and/or assessing, and for the administration of, cancer treatments involving one or more BET bromodomain inhibitors or BCL-xL inhibitors, or combinations thereof.

### BACKGROUND

The BET protein family consists of four polypeptides, BRD2, BRD3, BRD4 and BRDT, which encode tandem bromodomains that recognize acetylated lysine residues within histone and non-histone proteins. (Chiang, C. M. et al. The double bromodomain-containing chromatin adaptor Brd4 and transcriptional regulation. Wu S Y, J Biol Chem. 282, 13141-5, (2007)). BET proteins function as molecular adaptors, tethering transcriptional co-regulators to specific genomic locations marked by histone acetylation. Small molecule inhibition of BET bromodomains results in the suppression of a circumscribed set of genes, including key mediators of cellular transformation and cell fate. Among the genes impacted include the oncogenic transcription factors MYC and MYB, as well as members of the anti-apoptotic BCL-2 family. (Zuber, J. et al. RNAi screen identifies Brd4 as a therapeutic target in acute myeloid leukaemia. Nature. 478, 524-8, (2011); Delmore, J. E. et al. BET bromodomain inhibition as a therapeutic strategy to target c-Myc. Cell. 16, 904-17, (2011); Dawson, M. A. et al. Inhibition of BET recruitment to chromatin as an effective treatment for MLL-fusion leukaemia. Nature. 478, 529-33, (2011); Mertz, J. A. et. al. Targeting MYC dependence in cancer by inhibiting BET bromodomains. Proc Natl Acad Sci USA. 108, 16669-74, (2011)). However, the genes most altered by BET bromodomain inhibition vary across cell lines, cancer sub-type, and even within the same cell line over time.

While there are common transcriptional pathways affected by BET bromodomain inhibition in different contexts, the phenotypic responses to BET inhibition are not always governed by the same pathways. In addition, treatment of cancer cell lines with BET bromodomain inhibitors elicits a range of phenotypic effects, such as cellular differentiation, senescence, and apoptosis. This molecular heterogeneity creates a challenge when attempting to identify patients that will best benefit from this mechanism.

BET inhibitors have been established to induce growth inhibition of a wide range of tumor cell lines, and this growth inhibitory effect has been primarily attributed to G0/G1 phase cell cycle arrest, apoptosis, or some combination thereof. (Zuber, J. et al. RNAi screen identifies Brd4 as a therapeutic target in acute myeloid leukaemia. Nature. 478, 524-8, (2011); Delmore, J. E. et al. BET bromodomain inhibition as a therapeutic strategy to target c-Myc. Cell. 16, 904-17, (2011); Dawson, M. A. et al. Inhibition of BET recruitment to chromatin as an effective treatment for MLL-fusion leukaemia. Nature. 478, 529-33, (2011); Mertz, J. A. et. al. Targeting MYC dependence in cancer by inhibiting BET bromodomains. Proc Natl Acad Sci US A. 108, 16669-74, (2011)). Until now, however, specific correlations between cancer cell phenotypes and BET inhibitor selectivity have not been realized. Such determinations would assist in developing e.g., more systematic patient screening/selection processes, improved compositions for targeting BET bromodomains, and robust criteria for assessing patient qualifications to other anti-cancer therapies.

The instant disclosure fulfills such a need and relates to, in one aspect, the identification of BCL-xL as a marker for determining sensitivity to BET bromodomain inhibitors, and other therapeutic utilities and compositions resulting therefrom. The instant disclosure also related to, in another aspect, the identification of BCL-2, BCL-w, and Bcl-2-associated death promoter (BAD), each of which independently or in combination serve as markers for determining sensitivity to BET bromodomain inhibitors, and other therapeutic utilities and compositions resulting therefrom.

### SUMMARY

It has now been found that high basal expression of BCL-xL correlates with lower phenotypic sensitivity to BET bromodomain inhibition. See e.g., FIG. 9. It has also now been found that high basal expression of BCL2 gene, low basal expression of BCL2L2 gene, and low basal expression of BAD gene each correlate with higher phenotypic sensitivity to BET bromodomain inhibition. See e.g., FIG. 9.

Methods for treating subjects with a cancer which is not overexpressing BCL-xL have now been found. Such methods include e.g., administering an effective amount of a BET bromodomain inhibitor to the subject. Methods for treating subjects with a cancer which is overexpressing BCL-2, not overexpressing BCL-w, or not overexpressing BAD, independently or in combination with one another, have also now been found. Such methods also include e.g., administering an effective amount of a BET bromodomain inhibitor to the subject.

Also provided herein are methods for treating a subject with a cancer, comprising determining the expression levels of BCL-xL in the subject's cancer and administering to the subject an effective amount of a BET bromodomain inhibitor, if the subject's cancer is not overexpressing BCL-xL.

Further provided are methods for treating a subject with a cancer, comprising determining the expression levels of BCL-2 alone or in combination with the expression levels of one or more of BCL-xL, BCL-w, or BAD, in the subject's cancer and administering to the subject an effective amount of a BET bromodomain inhibitor, if the subject's cancer is overexpressing BCL-2. Also further provided are methods for treating a subject with a cancer, comprising determining the expression levels of BCL-w alone or in combination with the expression levels of one or more of BCL-xL, BCL-2, or BAD, in the subject's cancer and administering to the subject an effective amount of a BET bromodomain inhibitor, if the subject's cancer is not overexpressing BCL-w. Also further provided are methods for treating a subject with a cancer, comprising determining the expression levels of BAD alone or in combination with the expression levels of one or more of BCL-xL, BCL-2, or BCL-w in the subject's cancer and administering to the subject an effective amount of a BET bromodomain inhibitor, if the subject's cancer is not overexpressing BAD.

Also provided are methods for assessing the efficacy of a BET inhibitor to treat cancer in a patient comprising obtaining a sample of the cancer from the patient and determining the expression level of BCL-xL in the cancer, wherein the BET inhibitor is likely to be effective if the BCL-xL is not overexpressed.

Further provided are methods for assessing the efficacy of a BET inhibitor to treat cancer in a patient comprising obtaining a sample of the cancer from the patient and determining the expression level of BCL-2, alone or in combination with the expression levels of one or more of BCL-xL, BCL-w, or BAD, in the cancer, wherein the BET inhibitor is likely to be effective if the BCL-2 gene is overexpressed. Also further provided are methods for assessing the efficacy of a BET inhibitor to treat cancer in a patient comprising obtaining a sample of the cancer from the patient and determining the expression level of BCL-w, alone or in combination with the expression levels of one or more of BCL-xL, BCL-2, or BAD, in the cancer, wherein the BET inhibitor is likely to be effective if the BCL-w gene is not overexpressed. Also further provided are methods for assessing the efficacy of a BET inhibitor to treat cancer in a patient comprising obtaining a sample of the cancer from the patient and determining the expression level of BAD, alone or in combination with the expression levels of one or more of BCL-xL, BCL-2, or BCL-w, in the cancer, wherein the BET inhibitor is likely to be effective if the BAD gene is not overexpressed.

Also provided are methods of determining further treatments for patients currently being treated with a BET inhibitor for cancer comprising the steps of obtaining a sample of the cancer from the patient; and determining if BCL-xL is overexpressed in the cancer, wherein treatment with a BET inhibitor is likely to be continued (or is continued) if BCL-xL is not overexpressed.

Further provided are methods of determining further treatments for patients currently being treated with a BET inhibitor for cancer comprising the steps of obtaining a sample of the cancer from the patient; and determining if BCL-2 is overexpressed, alone or in combination with the expression levels of one or more of BCL-xL, BCL-w, or BAD, in the cancer, wherein treatment with a BET inhibitor is likely to be continued (or is continued) if BCL-2 is overexpressed. Also further provided are methods of determining further treatments for patients currently being treated with a BET inhibitor for cancer comprising the steps of obtaining a sample of the cancer from the patient; and determining if BCL-w is overexpressed, alone or in combination with the expression levels of one or more of BCL-xL, BCL-2, or BAD, in the cancer, wherein treatment with a BET inhibitor is likely to be continued (or is continued) if BCL-w is not overexpressed. Also further provided are methods of determining further treatments for patients currently being treated with a BET inhibitor for cancer comprising the steps of obtaining a sample of the cancer from the patient; and determining if BAD is overexpressed, alone or in combination with the expression levels of one or more of BCL-xL, BCL-2, or BCL-w, in the cancer, wherein treatment with a BET inhibitor is likely to be continued (or is continued) if BAD is not overexpressed.

Also provided herein are methods for treating a subject with a cancer, comprising a combination therapy of an effective amount of a BET bromodomain inhibitor together with an effective amount of a BCL-xL inhibitor. This combination therapy is particularly suited to subjects whose cancer is overexpressing BCL-xL.

Also provided herein are packaged composition comprising an effective amount of a BET inhibitor; and a pharmaceutically acceptable carrier or diluent, wherein the composition is packaged with instructions to treat a subject suffering from a cancer that is not overexpressing at least one of BCL-xL, BCL-w, and BAD, and optionally overexpressing BCL-2.

### BRIEF DESCRIPTION OF THE FIGURES

FIG. 1 represents a comparison of phenotypic response to certain BET inhibitors.

FIG. 2 represents a correlation between apoptosis and sensitivity to BET inhibition, and further demonstrates the modulation of the expression of apoptotic factors following treatment with a BET inhibitor.

FIG. 3 illustrates a response of BET inhibitor resistant A375 cells and demonstrates the increased expression of BCL2L1 (coding for BCL-xL) in the resistant cells.

FIG. 4 illustrates that knockdown of BCL-xL with RNAi restores sensitivity and that ectopic expression of BCL-xL abrogates the apoptotic response to BET inhibition in parental A375 cells.

FIG. 5 illustrates that pharmacological inhibition of BCL-xL restores sensitivity to BET inhibition in resistant A375 cells.

FIG. 6 represents the characterization of NOMO-1 cells engineered to be resistant to BET inhibition where a) illustrates the proliferation of parental and resistant cells in the presence of a BET inhibitor; b) illustrates a muted apoptotic response to a BET inhibitor in resistant cells; c) illustrates increased BCL2 expression in resistant NOMO-1 cells treated with BET inhibitor and increased BCL2L11 expression in both resistant and parental NOMO-1 cells; d) illustrates a reduced BCL2L11/BCL2 ratio in resistant NOMO-1 cells; e) illustrates reduced viability and enhanced apoptosis in resistant cells treated with RNAi to BCL2; and f) illustrates increased phenotypic sensitivity to the BCL2 inhibitor ABT-199 in resistant NOMO-1 cells.

FIG. 7 indicates tumor subtypes of the 245 cell lines used for comparison of gene expression and phenotypic response to BET inhibition.

FIG. 8 illustrates differential expression of indicated genes in sensitive vs. insensitive cell lines.

FIG. 9 illustrates expression of BCL2, BCL2L1, BCL2L1, BAD, and BCL2AF1 in BETi sensitive vs. insensitive cell lines.

FIG. 10 illustrates that high expression of BCL2 and low expression of BCL2L2, BCL2L1, or BAD are correlated with sensitivity to BET inhibition.

FIG. 11 illustrates high expression of BCL2 and low expression of other factors enhances prediction of phenotypic response to BET inhibition.

FIG. 12 illustrates data as in FIG. 10, but with cell lines of hematopoetic origin.

FIG. 13 illustrates data as in FIG. 11, but with cell lines of hematopoetic origin.

FIG. 14 illustrates data as in FIG. 10, but with cell lines of solid tumor origin.

FIG. 15 illustrates data as in FIG. 11, but with cell lines of solid tumor origin.

FIG. 16 represents the enrichment of cancer subtypes with high response rate to BET inhibition following selection by high expression of BCL2 and low expression of BCL2L1 or BAD.

FIG. 17 illustrates BCL2 and BCL2L1 expression as determined by q-RTPCR can be used to predict cell line response to BET inhibition.

### DETAILED DESCRIPTION

It has now been found that the expression/overexpression of at least four markers (BCL-xL, BCL-2, BCL-w, and BAD) correlate with the effectiveness of BET inhibitors. Based on this discovery, methods of treating cancers in patients with BET inhibitors based on the expression/overexpression of such markers are described below. Expression/overexpression of these markers in the disclosed cancer treatments can be alone or in combination with one or more of the other markers described herein.

In one aspect, the present disclosure provides a method of treating a subject (e.g., a human) with a cancer which is not overexpressing BCL-xL comprising administering to the subject an effective amount of a BET bromodomain inhibitor. In an alternative, the present disclosure provides a method of treating a subject (e.g., a human) with a cancer which is not overexpressing BCL-xL comprising administering to the subject an effective amount of a BET bromodomain inhibitor, wherein prior to treatment, the cancer has been determined to not be overexpressing BCL-xL.

In another aspect, the present disclosure provides a method of treating a subject (e.g., a human) with a cancer which is overexpressing BCL-2 comprising administering to the subject an effective amount of a BET bromodomain inhibitor. In an alternative, the present disclosure provides a method of treating a subject (e.g., a human) with a cancer which is overexpressing BCL-2 comprising administering to the subject an effective amount of a BET bromodomain inhibitor, wherein prior to treatment, the cancer has been determined to be overexpressing BCL-2.

In another aspect, the present disclosure provides a method of treating a subject (e.g., a human) with a cancer which is not overexpressing BCL-w comprising administering to the subject an effective amount of a BET bromodomain inhibitor. In an alternative, the present disclosure provides a method of treating a subject (e.g., a human) with a cancer which is not overexpressing BCL-w comprising administering to the subject an effective amount of a BET bromodomain inhibitor, wherein prior to treatment, the cancer has been determined to not be overexpressing BCL-w.

In another aspect, the present disclosure provides a method of treating a subject (e.g., a human) with a cancer which is not overexpressing BAD comprising administering to the subject an effective amount of a BET bromodomain inhibitor. In an alternative, the present disclosure provides a method of treating a subject (e.g., a human) with a cancer which is not overexpressing BAD comprising administering to the subject an effective amount of a BET bromodomain inhibitor, wherein prior to treatment, the cancer has been determined to not be overexpressing BAD.

In another aspect, the present disclosure provides a method of treating a subject (e.g., a human) with a cancer which is not overexpressing BCL-w and/or BAD, and/or overexpressing BCL-2, comprising administering to the subject an effective amount of a BET bromodomain inhibitor. In an alternative, the present disclosure provides a method of treating a subject (e.g., a human) with a cancer which is not overexpressing BCL-w and/or BAD, and/or overexpressing BCL-2, comprising administering to the subject an effective amount of a BET bromodomain inhibitor, wherein prior to treatment, the cancer has been determined to not be overexpressing BCL-w and/or BAD, and/or overexpressing BCL-2.

In another aspect, the present disclosure provides a method of treating a subject (e.g., a human) with a cancer which is not overexpressing BCL-xL and overexpressing BCL-2 comprising administering to the subject an effective amount of a BET bromodomain inhibitor.

In another aspect, the present disclosure provides a method of treating a subject (e.g., a human) with a cancer which is not overexpressing BCL-xL and BCL-w comprising administering to the subject an effective amount of a BET bromodomain inhibitor.

In another aspect, the present disclosure provides a method of treating a subject (e.g., a human) with a cancer which is not overexpressing BCL-xL and BAD comprising administering to the subject an effective amount of a BET bromodomain inhibitor.

In another aspect, the present disclosure provides a method of treating a subject (e.g., a human) with a cancer which is overexpressing BCL-2 and not overexpressing BCL-w comprising administering to the subject an effective amount of a BET bromodomain inhibitor.

In another aspect, the present disclosure provides a method of treating a subject (e.g., a human) with a cancer which is overexpressing BCL-2 and not overexpressing BAD comprising administering to the subject an effective amount of a BET bromodomain inhibitor.

In another aspect, the present disclosure provides a method of treating a subject (e.g., a human) with a cancer which is not overexpressing BCL-w and BAD comprising administering to the subject an effective amount of a BET bromodomain inhibitor.

In another aspect, the present disclosure provides a method of treating a subject (e.g., a human) with a cancer which is not overexpressing BCL-xL and BCL-w, and overexpressing BCL-2 comprising administering to the subject an effective amount of a BET bromodomain inhibitor.

In another aspect, the present disclosure provides a method of treating a subject (e.g., a human) with a cancer which is not overexpressing BCL-xL and BAD, and overexpressing BCL-2 comprising administering to the subject an effective amount of a BET bromodomain inhibitor.

In another aspect, the present disclosure provides a method of treating a subject (e.g., a human) with a cancer which is not overexpressing BCL-xL, not overexpressing BCL-w and BAD comprising administering to the subject an effective amount of a BET bromodomain inhibitor.

In another aspect, the present disclosure provides a method of treating a subject (e.g., a human) with a cancer which is overexpressing BCL-2, not overexpressing BCL-w and BAD comprising administering to the subject an effective amount of a BET bromodomain inhibitor.

In another aspect, the present disclosure provides a method of treating a subject (e.g., a human) with a cancer which is overexpressing BCL-2 and not overexpressing BCL-xL, BCL-w, and BAD comprising administering to the subject an effective amount of a BET bromodomain inhibitor.

The disclosed methods can include the step of assessing or determining the expression levels of one or more of BCL-xL, BCL-2, BCL-w, and BAD in order to select for patients likely to respond to treatment with a BET inhibitor. Methods incorporating such steps are described for example below.

In another aspect, the present disclosure also provides a method of treating a subject (e.g., a human) with a cancer, comprising determining the expression levels of BCL-xL of the subject's cancer and administering to the subject an effective amount of a BET bromodomain inhibitor, if the subject's cancer is not overexpressing BCL-xL. Alternatively, the present disclosure also provides for determining the expression levels of BCL-xL of the subject's cancer and administering an effective amount of a cancer therapy other than a BET bromodomain inhibitor, if the subject's cancer is overexpressing BCL-xL. In another aspect, the present disclosure also provides a method of treating a subject (e.g., a human) with a cancer, comprising determining the expression levels of BCL-xL of the subject's cancer and administering to the subject an effective amount of a BET bromodomain inhibitor, if the subject's cancer is not overexpressing BCL-xL, and determining the expression levels of BCL-xL of the subject's cancer and administering an effective amount of a cancer therapy other than a BET bromodomain inhibitor, if the subject's cancer is overexpressing BCL-xL.

In another aspect, the present disclosure also provides a method of treating a subject (e.g., a human) with a cancer, comprising determining the expression levels of BCL-2 of the subject's cancer and administering to the subject an effective amount of a BET bromodomain inhibitor, if the subject's cancer is overexpressing BCL-2. Alternatively, the present disclosure also provides for determining the expression levels of BCL-2 of the subject's cancer and administering an effective amount of a cancer therapy other than a BET bromodomain inhibitor, if the subject's cancer is not overexpressing BCL-2. In another aspect, the present disclosure also provides a method of treating a subject (e.g., a human) with a cancer, comprising determining the expression levels of BCL-2 of the subject's cancer and administering to the subject an effective amount of a BET bromodomain inhibitor, if the subject's cancer is overexpressing BCL-2, and determining the expression levels of BCL-2 of the subject's cancer and administering an effective amount of a cancer therapy other than a BET bromodomain inhibitor, if the subject's cancer is not overexpressing BCL-2.

In another aspect, the present disclosure also provides a method of treating a subject (e.g., a human) with a cancer, comprising determining the expression levels of BCL-w of the subject's cancer and administering to the subject an effective amount of a BET bromodomain inhibitor, if the subject's cancer is not overexpressing BCL-w. Alternatively, the present disclosure also provides for determining the expression levels of BCL-w of the subject's cancer and administering an effective amount of a cancer therapy other than a BET bromodomain inhibitor, if the subject's cancer is overexpressing BCL-w. In another aspect, the present disclosure also provides a method of treating a subject (e.g., a human) with a cancer, comprising determining the expression levels of BCL-w of the subject's cancer and administering to the subject an effective amount of a BET bromodomain inhibitor, if the subject's cancer is not overexpressing BCL-w, and determining the expression levels of BCL-w of the subject's cancer and administering an effective amount of a cancer therapy other than a BET bromodomain inhibitor, if the subject's cancer is overexpressing BCL-w.

In another aspect, the present disclosure also provides a method of treating a subject (e.g., a human) with a cancer, comprising determining the expression levels of BAD of the subject's cancer and administering to the subject an effective amount of a BET bromodomain inhibitor, if the subject's cancer is not overexpressing BAD. Alternatively, the present disclosure also provides for determining the expression levels of BAD of the subject's cancer and administering an effective amount of a cancer therapy other than a BET bromodomain inhibitor, if the subject's cancer is overexpressing BAD. In another aspect, the present disclosure also provides a method of treating a subject (e.g., a human) with a cancer, comprising determining the expression levels of BAD of the subject's cancer and administering to the subject an effective amount of a BET bromodomain inhibitor, if the subject's cancer is not overexpressing BAD, and determining the expression levels of BAD of the subject's cancer and administering an effective amount of a cancer therapy other than a BET bromodomain inhibitor, if the subject's cancer is overexpressing BAD.

In another aspect, the present disclosure also provides a method of treating a subject (e.g., a human) with a cancer, comprising determining the expression levels of BCL-xL and BCL-2 of the subject's cancer and administering to the subject an effective amount of a BET bromodomain inhibitor, if the subject's cancer is not overexpressing BCL-xL and overexpressing BCL-2. Alternatively, the present disclosure also provides for determining the expression levels of BCL-xL and BCL-2 of the subject's cancer and administering an effective amount of a cancer therapy other than a BET bromodomain inhibitor, if the subject's cancer is overexpressing BCL-xL and not overexpressing BCL-2. In another aspect, the present disclosure also provides a method of treating a subject (e.g., a human) with a cancer, comprising determining the expression levels of BCL-xL and BCL-2 of the subject's cancer and administering to the subject an effective amount of a BET bromodomain inhibitor, if the subject's cancer is not overexpressing BCL-xL and overexpressing BCL-2, and determining the expression levels of BCL-xL and BCL-2 of the subject's cancer and administering an effective amount of a cancer therapy other than a BET bromodomain inhibitor, if the subject's cancer is overexpressing BCL-xL and not overexpressing BCL-2.

In another aspect, the present disclosure also provides a method of treating a subject (e.g., a human) with a cancer, comprising determining the expression levels of BCL-xL and BCL-w of the subject's cancer and administering to the subject an effective amount of a BET bromodomain inhibitor, if the subject's cancer is not overexpressing BCL-xL and BCL-w. Alternatively, the present disclosure also provides for determining the expression levels of BCL-xL and BCL-w of the subject's cancer and administering an effective amount of a cancer therapy other than a BET bromodomain inhibitor, if the subject's cancer is overexpressing BCL-xL and BCL-w. In another aspect, the present disclosure also provides a method of treating a subject (e.g., a human) with a cancer, comprising determining the expression levels of BCL-xL and BCL-w of the subject's cancer and administering to the subject an effective amount of a BET bromodomain inhibitor, if the subject's cancer is not overexpressing BCL-xL and BCL-w, and determining the expression levels of BCL-xL and BCL-w of the subject's cancer and administering an effective amount of a cancer therapy other than a BET bromodomain inhibitor, if the subject's cancer is overexpressing BCL-xL and BCL-w.

In another aspect, the present disclosure also provides a method of treating a subject (e.g., a human) with a cancer, comprising determining the expression levels of BCL-xL and BAD of the subject's cancer and administering to the subject an effective amount of a BET bromodomain inhibitor, if the subject's cancer is not overexpressing BCL-xL and BAD. Alternatively, the present disclosure also provides for determining the expression levels of BCL-xL and BAD of the subject's cancer and administering an effective amount of a cancer therapy other than a BET bromodomain inhibitor, if the subject's cancer is overexpressing BCL-xL and BAD. In another aspect, the present disclosure also provides a method of treating a subject (e.g., a human) with a cancer, comprising determining the expression levels of BCL-xL and BAD of the subject's cancer and administering to the subject an effective amount of a BET bromodomain inhibitor, if the subject's cancer is not overexpressing BCL-xL and BAD, and determining the expression levels of BCL-xL and BAD of the subject's cancer and administering an effective amount of a cancer therapy other than a BET bromodomain inhibitor, if the subject's cancer is overexpressing BCL-xL and BAD.

In another aspect, the present disclosure also provides a method of treating a subject (e.g., a human) with a cancer, comprising determining the expression levels of BCL-2 and BCL-w of the subject's cancer and administering to the subject an effective amount of a BET bromodomain inhibitor, if the subject's cancer is overexpressing BCL-2 and not overexpressing BCL-w. Alternatively, the present disclosure also provides for determining the expression levels of BCL-2 and BCL-w of the subject's cancer and administering an effective amount of a cancer therapy other than a BET bromodomain inhibitor, if the subject's cancer is not overexpressing BCL-2 and overexpressing BCL-w. In another aspect, the present disclosure also provides a method of treating a subject (e.g., a human) with a cancer, comprising determining the expression levels of BCL-2 and BCL-w of the subject's cancer and administering to the subject an effective amount of a BET bromodomain inhibitor, if the subject's cancer is overexpressing BCL-2 and not overexpressing BCL-w, and determining the expression levels of BCL-2 and BCL-w of the subject's cancer and administering an effective amount of a cancer therapy other than a BET bromodomain inhibitor, if the subject's cancer is not overexpressing BCL-2 and overexpressing BCL-w.

In another aspect, the present disclosure also provides a method of treating a subject (e.g., a human) with a cancer, comprising determining the expression levels of BCL-2 and BAD of the subject's cancer and administering to the subject an effective amount of a BET bromodomain inhibitor, if the subject's cancer is overexpressing BCL-2 and not overexpressing BAD. Alternatively, the present disclosure also provides for determining the expression levels of BCL-2 and BAD of the subject's cancer and administering an effective amount of a cancer therapy other than a BET bromodomain inhibitor, if the subject's cancer is not overexpressing BCL-2 and overexpressing BAD. In another aspect, the present disclosure also provides a method of treating a subject (e.g., a human) with a cancer, comprising determining the expression levels of BCL-2 and BAD of the subject's cancer and administering to the subject an effective amount of a BET bromodomain inhibitor, if the subject's cancer is overexpressing BCL-2 and not overexpressing BAD, and determining the expression levels of BCL-2 and BAD of the subject's cancer and administering an effective amount of a cancer therapy other than a BET bromodomain inhibitor, if the subject's cancer is not overexpressing BCL-2 and overexpressing BAD.

In another aspect, the present disclosure also provides a method of treating a subject (e.g., a human) with a cancer, comprising determining the expression levels of BCL-w and BAD of the subject's cancer and administering to the subject an effective amount of a BET bromodomain inhibitor, if the subject's cancer is not overexpressing BCL-w and BAD. Alternatively, the present disclosure also provides for determining the expression levels of BCL-w and BAD of the subject's cancer and administering an effective amount of a cancer therapy other than a BET bromodomain inhibitor, if the subject's cancer is overexpressing BCL-w and BAD. In another aspect, the present disclosure also provides a method of treating a subject (e.g., a human) with a cancer, comprising determining the expression levels of BCL-w and BAD of the subject's cancer and administering to the subject an effective amount of a BET bromodomain inhibitor, if the subject's cancer is not overexpressing BCL-w and BAD, and determining the expression levels of BCL-w and BAD of the subject's cancer and administering an effective amount of a cancer therapy other than a BET bromodomain inhibitor, if the subject's cancer is overexpressing BCL-w and BAD.

In another aspect, the present disclosure also provides a method of treating a subject (e.g., a human) with a cancer, comprising determining the expression levels of BCL-xL, BCL-2, and BCL-w of the subject's cancer and administering to the subject an effective amount of a BET bromodomain inhibitor, if the subject's cancer is not overexpressing BCL-xL and BCL-w, and overexpressing BCL-2. Alternatively, the present disclosure also provides for determining the expression levels of BCL-xL, BCL-2, and BCL-w of the subject's cancer and administering an effective amount of a cancer therapy other than a BET bromodomain inhibitor, if the subject's cancer is overexpressing BCL-xL and BCL-w, and not overexpressing BCL-2. In another aspect, the present disclosure also provides a method of treating a subject (e.g., a human) with a cancer, comprising determining the expression levels of BCL-xL, BCL-2, and BCL-w of the subject's cancer and administering to the subject an effective amount of a BET bromodomain inhibitor, if the subject's cancer is not overexpressing BCL-xL and BCL-w, and overexpressing BCL-2, and determining the expression levels of BCL-xL, BCL-2, and BCL-w of the subject's cancer and administering an effective amount of a cancer therapy other than a BET bromodomain inhibitor, if the subject's cancer is overexpressing BCL-xL and BCL-w, and not overexpressing BCL-2.

In another aspect, the present disclosure also provides a method of treating a subject (e.g., a human) with a cancer, comprising determining the expression levels of BCL-xL, BCL-2, and BAD of the subject's cancer and administering to the subject an effective amount of a BET bromodomain inhibitor, if the subject's cancer is not overexpressing BCL-xL and BAD, and overexpressing BCL-2. Alternatively, the present disclosure also provides for determining the expression levels of BCL-xL, BCL-2, and BAD of the subject's cancer and administering an effective amount of a cancer therapy other than a BET bromodomain inhibitor, if the subject's cancer is overexpressing BCL-xL and BAD, and not overexpressing BCL-2. In another aspect, the present disclosure also provides a method of treating a subject (e.g., a human) with a cancer, comprising determining the expression levels of BCL-xL, BCL-2, and BAD of the subject's cancer and administering to the subject an effective amount of a BET bromodomain inhibitor, if the subject's cancer is not overexpressing BCL-xL and BAD, and overexpressing BCL-2, and determining the expression levels of BCL-xL, BCL-2, and BAD of the subject's cancer and administering an effective amount of a cancer therapy other than a BET bromodomain inhibitor, if the subject's cancer is overexpressing BCL-xL and BAD, and not overexpressing BCL-2.

In another aspect, the present disclosure also provides a method of treating a subject (e.g., a human) with a cancer, comprising determining the expression levels of BCL-xL, BCL-w, and BAD of the subject's cancer and administering to the subject an effective amount of a BET bromodomain inhibitor, if the subject's cancer is not overexpressing BCL-xL, BCL-w, and BAD. Alternatively, the present disclosure also provides for determining the expression levels of BCL-xL, BCL-w, and BAD of the subject's cancer and administering an effective amount of a cancer therapy other than a BET bromodomain inhibitor, if the subject's cancer is overexpressing BCL-xL, BCL-w, and BAD. In another aspect, the present disclosure also provides a method of treating a subject (e.g., a human) with a cancer, comprising determining the expression levels of BCL-xL, BCL-w, and BAD of the subject's cancer and administering to the subject an effective amount of a BET bromodomain inhibitor, if the subject's cancer is not overexpressing BCL-xL, BCL-w, and BAD, and determining the expression levels of BCL-xL, BCL-w, and BAD of the subject's cancer and administering an effective amount of a cancer therapy other than a BET bromodomain inhibitor, if the subject's cancer is overexpressing BCL-xL, BCL-w, and BAD.

In another aspect, the present disclosure also provides a method of treating a subject (e.g., a human) with a cancer, comprising determining the expression levels of BCL-2, BCL-w, and BAD of the subject's cancer and administering to the subject an effective amount of a BET bromodomain inhibitor, if the subject's cancer is overexpressing BCL-2, and not overexpressing BCL-w and BAD. Alternatively, the present disclosure also provides for determining the expression levels of BCL-2, BCL-w, and BAD of the subject's cancer and administering an effective amount of a cancer therapy other than a BET bromodomain inhibitor, if the subject's cancer is overexpressing BCL-w and BAD, and not overexpressing BCL-2. In another aspect, the present disclosure also provides a method of treating a subject (e.g., a human) with a cancer, comprising determining the expression levels of BCL-2, BCL-w, and BAD of the subject's cancer and administering to the subject an effective amount of a BET bromodomain inhibitor, if the subject's cancer is overexpressing BCL-2, and not overexpressing BCL-w and BAD, and determining the expression levels of BCL-2, BCL-w, and BAD of the subject's cancer and administering an effective amount of a cancer therapy other than a BET bromodomain inhibitor, if the subject's cancer is overexpressing BCL-w and BAD, and not overexpressing BCL-2.

In another aspect, the present disclosure also provides a method of treating a subject (e.g., a human) with a cancer, comprising determining the expression levels of BCL-xL, BCL-2, BCL-w, and BAD of the subject's cancer and administering to the subject an effective amount of a BET bromodomain inhibitor, if the subject's cancer is overexpressing BCL-2, and not overexpressing BCL-xL, BCL-w, and BAD. Alternatively, the present disclosure also provides for determining the expression levels of BCL-xL, BCL-2, BCL-w, and BAD of the subject's cancer and administering an effective amount of a cancer therapy other than a BET bromodomain inhibitor, if the subject's cancer is overexpressing BCL-xL, BCL-w, and BAD, and not overexpressing BCL-2. In another aspect, the present disclosure also provides a method of treating a subject (e.g., a human) with a cancer, comprising determining the expression levels of BCL-xL, BCL-2, BCL-w, and BAD of the subject's cancer and administering to the subject an effective amount of a BET bromodomain inhibitor, if the subject's cancer is overexpressing BCL-2, and not overexpressing BCL-xL, BCL-w, and BAD, and determining the expression levels of BCL-xL, BCL-2, BCL-w, and BAD of the subject's cancer and administering an effective amount of a cancer therapy other than a BET bromodomain inhibitor, if the subject's cancer is overexpressing BCL-xL, BCL-w, and BAD, and not overexpressing BCL-2.

In another aspect, the methods described herein further comprise determining further treatments for patients currently being treated with a BET inhibitor for cancer comprising the steps of obtaining a sample of the cancer from the patient; and determining if BCL-xL is overexpressed in the cancer, wherein treatment with a BET inhibitor is likely to be continued (or is continued) if BCL-xL is not overexpressed. Alternatively, treatment with a BET inhibitor is likely not to be continued (or not continued) if BCL-xL is overexpressed. In another alternative treatment with both a BET and BCL-xL inhibitor is likely to be used (or is used) if BCL-xL is overexpressed. In another aspect, the methods described herein further comprise determining further treatments for patients currently being treated with a BET inhibitor for cancer comprising the steps of obtaining a sample of the cancer from the patient; and determining if BCL-xL is overexpressed in the cancer, wherein treatment with a BET inhibitor is likely to be continued (or is continued) if BCL-xL is not overexpressed, and treatment with a BET inhibitor is likely not to be continued (or not continued) if BCL-xL is overexpressed, and treatment with both a BET and BCL-xL inhibitor is likely to be used (or is used) if BCL-xL is overexpressed.

In another aspect, the methods described herein further comprise determining further treatments for patients currently being treated with a BET inhibitor for cancer comprising the steps of obtaining a sample of the cancer from the patient; and determining if BCL-2 is not overexpressed in the cancer, wherein treatment with a BET inhibitor is likely to be continued (or is continued) if BCL-2 is overexpressed. Alternatively, treatment with a BET inhibitor is likely not to be continued (or not continued) if BCL-2 is not overexpressed. In another aspect, the methods described herein further comprise determining further treatments for patients currently being treated with a BET inhibitor for cancer comprising the steps of obtaining a sample of the cancer from the patient; and determining if BCL-2 is not overexpressed in the cancer, wherein treatment with a BET inhibitor is likely to be continued (or is continued) if BCL-2 is overexpressed, and treatment with a BET inhibitor is likely not to be continued (or not continued) if BCL-2 is not overexpressed.

In another aspect, the methods described herein further comprise determining further treatments for patients currently being treated with a BET inhibitor for cancer comprising the steps of obtaining a sample of the cancer from the patient; and determining if BCL-w is overexpressed in the cancer, wherein treatment with a BET inhibitor is likely to be continued (or is continued) if BCL-w is not overexpressed. Alternatively, treatment with a BET inhibitor is likely not to be continued (or not continued) if BCL-w is overexpressed. In another aspect, the methods described herein further comprise determining further treatments for patients currently being treated with a BET inhibitor for cancer comprising the steps of obtaining a sample of the cancer from the patient; and determining if BCL-w is overexpressed in the cancer, wherein treatment with a BET inhibitor is likely to be continued (or is continued) if BCL-w is not overexpressed, and treatment with a BET inhibitor is likely not to be continued (or not continued) if BCL-w is overexpressed.

It is also to be understood that BCL-xL inhibitors also act as BCL-w inhibitors. Thus, in another aspect, the methods described herein further comprise determining further treatments for patients currently being treated with a BET inhibitor for cancer comprising the steps of obtaining a sample of the cancer from the patient; and determining if BCL-w is overexpressed in the cancer, wherein treatment with a BET inhibitor is likely to be continued (or is continued) if both a BET and BCL-xL inhibitor is likely to be used (or is used) if BCL-w is overexpressed. In another aspect, the methods described herein further comprise determining further treatments for patients currently being treated with a BET inhibitor for cancer comprising the steps of obtaining a sample of the cancer from the patient; and determining if BCL-w is overexpressed in the cancer, wherein treatment with a BET inhibitor is likely to be continued (or is continued) if BCL-w is not overexpressed, and treatment with a BET inhibitor is likely not to be continued (or not continued) if BCL-w is overexpressed, and treatment with both a BET and BCL-w inhibitor is likely to be used (or is used) if BCL-w is overexpressed.

In another aspect, the methods described herein further comprise determining further treatments for patients currently being treated with a BET inhibitor for cancer comprising the steps of obtaining a sample of the cancer from the patient; and determining if BAD is overexpressed in the cancer, wherein treatment with a BET inhibitor is likely to be continued (or is continued) if BAD is not overexpressed. Alternatively, treatment with a BET inhibitor is likely not to be continued (or not continued) if BAD is overexpressed. In another aspect, the methods described herein further comprise determining further treatments for patients currently being treated with a BET inhibitor for cancer comprising the steps of obtaining a sample of the cancer from the patient; and determining if BAD is overexpressed in the cancer, wherein treatment with a BET inhibitor is likely to be continued (or is continued) if BAD is not overexpressed, and treatment with a BET inhibitor is likely not to be continued (or not continued) if BAD is overexpressed.

In another aspect, the methods described herein further comprise determining further treatments for patients currently being treated with a BET inhibitor for cancer comprising the steps of obtaining a sample of the cancer from the patient; and determining if BCL-xL and BAD are overexpressed in the cancer, wherein treatment with a BET inhibitor is likely to be continued (or is continued) if BCL-xL and BAD are not overexpressed. Alternatively, treatment with a BET inhibitor is likely not to be continued (or not continued) if BCL-xL and BAD are overexpressed. In another aspect, the methods described herein further comprise determining further treatments for patients currently being treated with a BET inhibitor for cancer comprising the steps of obtaining a sample of the cancer from the patient; and determining if BCL-xL and BAD are overexpressed in the cancer, wherein treatment with a BET inhibitor is likely to be continued (or is continued) if BCL-xL and BAD are not overexpressed, and treatment with a BET inhibitor is likely not to be continued (or not continued) if BCL-xL and BAD are overexpressed.

In another aspect, the methods described herein further comprise determining further treatments for patients currently being treated with a BET inhibitor for cancer comprising the steps of obtaining a sample of the cancer from the patient; and determining if BCL-xL and BCL-w are overexpressed in the cancer, wherein treatment with a BET inhibitor is likely to be continued (or is continued) if BCL-xL and BCL-w are not overexpressed. Alternatively, treatment with a BET inhibitor is likely not to be continued (or not continued) if BCL-xL and BCL-w are overexpressed. In another aspect, the methods described herein further comprise determining further treatments for patients currently being treated with a BET inhibitor for cancer comprising the steps of obtaining a sample of the cancer from the patient; and determining if BCL-xL and BCL-w are overexpressed in the cancer, wherein treatment with a BET inhibitor is likely to be continued (or is continued) if BCL-xL and BCL-w are not overexpressed, and treatment with a BET inhibitor is likely not to be continued (or not continued) if BCL-xL and BCL-w are overexpressed.

In another aspect, the methods described herein further comprise determining further treatments for patients currently being treated with a BET inhibitor for cancer comprising the steps of obtaining a sample of the cancer from the patient; and determining if BAD and BCL-w are overexpressed in the cancer, wherein treatment with a BET inhibitor is likely to be continued (or is continued) if BAD and BCL-w are not overexpressed. Alternatively, treatment with a BET inhibitor is likely not to be continued (or not continued) if BAD and BCL-w are overexpressed. In another aspect, the methods described herein further comprise determining further treatments for patients currently being treated with a BET inhibitor for cancer comprising the steps of obtaining a sample of the cancer from the patient; and determining if BAD and BCL-w are overexpressed in the cancer, wherein treatment with a BET inhibitor is likely to be continued (or is continued) if BAD and BCL-w are not overexpressed, and treatment with a BET inhibitor is likely not to be continued (or not continued) if BAD and BCL-w are overexpressed.

In another aspect, the methods described herein further comprise determining further treatments for patients currently being treated with a BET inhibitor for cancer comprising the steps of obtaining a sample of the cancer from the patient; and determining if BAD and BCL-w are overexpressed in the cancer and if BCL-2 is not overexpressed, wherein treatment with a BET inhibitor is likely to be continued (or is continued) if BAD and BCL-w are not overexpressed and BCL-2 is overexpressed. Alternatively, treatment with a BET inhibitor is likely not (or not continued) to be continued if BAD and BCL-w are overexpressed and BCL-2 is not overexpressed. In another aspect, the methods described herein further comprise determining further treatments for patients currently being treated with a BET inhibitor for cancer comprising the steps of obtaining a sample of the cancer from the patient; and determining if BAD and BCL-w are overexpressed in the cancer and if BCL-2 is not overexpressed, wherein treatment with a BET inhibitor is likely to be continued (or is continued) if BAD and BCL-w are not overexpressed and BCL-2 is overexpressed, and treatment with a BET inhibitor is likely not (or not continued) to be continued if BAD and BCL-w are overexpressed and BCL-2 is not overexpressed.

In another aspect, the methods described herein further comprise determining further treatments for patients currently being treated with a BET inhibitor for cancer comprising the steps of obtaining a sample of the cancer from the patient; and determining if BCL-xL and BCL-w are overexpressed in the cancer and if BCL-2 is not overexpressed, wherein treatment with a BET inhibitor is likely to be continued (or is continued) if BCL-xL and BCL-w are not overexpressed and BCL-2 is overexpressed. Alternatively, treatment with a BET inhibitor is likely not to be continued (or not continued) if BCL-xL and BCL-w are overexpressed and BCL-2 is not overexpressed. In another aspect, the methods described herein further comprise determining further treatments for patients currently being treated with a BET inhibitor for cancer comprising the steps of obtaining a sample of the cancer from the patient; and determining if BCL-xL and BCL-w are overexpressed in the cancer and if BCL-2 is not overexpressed, wherein treatment with a BET inhibitor is likely to be continued (or is continued) if BCL-xL and BCL-w are not overexpressed and BCL-2 is overexpressed, and treatment with a BET inhibitor is likely not to be continued (or not continued) if BCL-xL and BCL-w are overexpressed and BCL-2 is not overexpressed.

In another aspect, the methods described herein further comprise determining further treatments for patients currently being treated with a BET inhibitor for cancer comprising the steps of obtaining a sample of the cancer from the patient; and determining if BCL-xL and BAD are overexpressed in the cancer and if BCL-2 is not overexpressed, wherein treatment with a BET inhibitor is likely to be continued (or is continued) if BCL-xL and BAD are not overexpressed and BCL-2 is overexpressed. Alternatively, treatment with a BET inhibitor is likely not to be continued (or not continued) if BCL-xL and BAD are overexpressed and BCL-2 is not overexpressed. In another aspect, the methods described herein further comprise determining further treatments for patients currently being treated with a BET inhibitor for cancer comprising the steps of obtaining a sample of the cancer from the patient; and determining if BCL-xL and BAD are overexpressed in the cancer and if BCL-2 is not overexpressed, wherein treatment with a BET inhibitor is likely to be continued (or is continued) if BCL-xL and BAD are not overexpressed and BCL-2 is overexpressed, and treatment with a BET inhibitor is likely not to be continued (or not continued) if BCL-xL and BAD are overexpressed and BCL-2 is not overexpressed.

In another aspect, the methods described herein further comprise determining further treatments for patients currently being treated with a BET inhibitor for cancer comprising the steps of obtaining a sample of the cancer from the patient; and determining if BCL-xL, BCL-w, and BAD are overexpressed in the cancer and if BCL-2 is not overexpressed, wherein treatment with a BET inhibitor is likely to be continued (or is continued) if BCL-xL, BCL-w, and BAD are not overexpressed and BCL-2 is overexpressed. Alternatively, treatment with a BET inhibitor is likely not to be continued (or not continued) if BCL-xL, BCL-w, and BAD are overexpressed and BCL-2 is not overexpressed. In another aspect, the methods described herein further comprise determining further treatments for patients currently being treated with a BET inhibitor for cancer comprising the steps of obtaining a sample of the cancer from the patient; and determining if BCL-xL, BCL-w, and BAD are overexpressed in the cancer and if BCL-2 is not overexpressed, wherein treatment with a BET inhibitor is likely to be continued (or is continued) if BCL-xL, BCL-w, and BAD are not overexpressed and BCL-2 is overexpressed, and treatment with a BET inhibitor is likely not to be continued (or not continued) if BCL-xL, BCL-w, and BAD are overexpressed and BCL-2 is not overexpressed.

In another aspect, the present disclosure also provides a method of treating a subject (e.g., a human) with a cancer, comprising administering to the subject an effective amount of a BET bromodomain inhibitor and an effective amount of a BCL-xL inhibitor.

In another aspect, the present disclosure also provides a method of treating a subject (e.g., a human) with a BCL-2 overexpressing cancer, comprising administering to a subject in need thereof an effective amount of a BET bromodomain inhibitor.

In another aspect, the present disclosure also provides a method of treating a subject (e.g., a human) with a BCL-2 overexpressing cancer, comprising administering to the subject an effective amount of a BET bromodomain inhibitor and an effective amount of a BCL-xL inhibitor.

In another aspect, the present disclosure also provides a method of treating a subject (e.g., a human) with a BCL-xL not overexpressing cancer, comprising administering to a subject in need thereof an effective amount of a BET bromodomain inhibitor.

In another aspect, the present disclosure also provides a method of treating a subject (e.g., a human) with a BCL-xL overexpressing cancer, comprising administering to the subject an effective amount of a BET bromodomain inhibitor and an effective amount of a BCL-xL inhibitor.

In another aspect, the present disclosure also provides a method of treating a subject (e.g., a human) with a BCL-w not overexpressing cancer, comprising administering to a subject in need thereof an effective amount of a BET bromodomain inhibitor.

In another aspect, the present disclosure also provides a method of treating a subject (e.g., a human) with a BCL-w overexpressing cancer, comprising administering to the subject an effective amount of a BET bromodomain inhibitor and an effective amount of a BCL-xL inhibitor.

In another aspect, the present disclosure also provides a method of treating a subject (e.g., a human) with a BAD not overexpressing cancer, comprising administering to a subject in need thereof an effective amount of a BET bromodomain inhibitor.

In another aspect, the present disclosure also provides a method of treating a subject (e.g., a human) with a BCL-xL and BAD not overexpressing cancer, comprising administering to a subject in need thereof an effective amount of a BET bromodomain inhibitor.

In another aspect, the present disclosure also provides a method of treating a subject (e.g., a human) with a BCL-xL and BAD overexpressing cancer, comprising administering to the subject an effective amount of a BET bromodomain inhibitor and an effective amount of a BCL-xL inhibitor.

In another aspect, the present disclosure also provides a method of treating a subject (e.g., a human) with a BCL-xL and BCL2L2 not overexpressing cancer, comprising administering to a subject in need thereof an effective amount of a BET bromodomain inhibitor.

In another aspect, the present disclosure also provides a method of treating a subject (e.g., a human) with a BCL-xL and BCL-w overexpressing cancer, comprising administering to the subject an effective amount of a BET bromodomain inhibitor and an effective amount of a BCL-xL inhibitor.

In another aspect, the present disclosure also provides a method of treating a subject (e.g., a human) with a BCL-xL and BCL-w overexpressing cancer, comprising administering to the subject an effective amount of a BET bromodomain inhibitor and an effective amount of a BCL-xL inhibitor.

In another aspect, the present disclosure also provides a method of treating a subject (e.g., a human) with a BAD and BCL-w not overexpressing cancer, comprising administering to a subject in need thereof an effective amount of a BET bromodomain inhibitor.

In another aspect, the present disclosure also provides a method of treating a subject (e.g., a human) with a BAD and BCL-w overexpressing cancer, comprising administering to the subject an effective amount of a BET bromodomain inhibitor and an effective amount of a BCL-xL inhibitor.

In another aspect, the present disclosure also provides a method of treating a subject (e.g., a human) with a BCL-xL, BAD, and BCL-w not overexpressing cancer, comprising administering to a subject in need thereof an effective amount of a BET bromodomain inhibitor.

In another aspect, the present disclosure also provides a method of treating a subject (e.g., a human) with a BCL-xL, BAD, and BCL-w overexpressing cancer, comprising administering to the subject an effective amount of a BET bromodomain inhibitor and an effective amount of a BCL-xL inhibitor.

Also provided herein are packaged compositions comprising a therapeutically effective amount of a BET inhibitor; and a pharmaceutically acceptable carrier or diluent, wherein the composition is packaged with instructions to treat a subject suffering from a cancer that is not overexpressing BCL-xL.

Also provided herein are packaged compositions comprising a therapeutically effective amount of a BET inhibitor; and a pharmaceutically acceptable carrier or diluent, wherein the composition is packaged with instructions to treat a subject suffering from a cancer that is not overexpressing BCL-w.

Also provided herein are packaged compositions comprising a therapeutically effective amount of a BET inhibitor; and a pharmaceutically acceptable carrier or diluent, wherein the composition is packaged with instructions to treat a subject suffering from a cancer that is not overexpressing BAD.

Also provided herein are packaged compositions comprising a therapeutically effective amount of a BET inhibitor; and a pharmaceutically acceptable carrier or diluent, wherein the composition is packaged with instructions to treat a subject suffering from a cancer that is overexpressing BCL-2.

Also provided herein are packaged compositions comprising a therapeutically effective amount of a BET inhibitor; and a pharmaceutically acceptable carrier or diluent, wherein the composition is packaged with instructions to treat a subject suffering from a cancer that is not overexpressing BCL-xL and overexpressing BCL-2; or not overexpressing BCL-xL and BCL-w; or not overexpressing BCL-xL and BAD; or overexpressing BCL-2 and not overexpressing BCL-w; or overexpressing BCL2 and not overexpressing BAD; or not overexpressing BCL-w and BAD; or not overexpressing BCL-xL and BCL-w, and overexpressing BCL-2; or not overexpressing BCL-xL and BAD, and overexpressing BCL-2; or not overexpressing BCL-xL, BCL-w, and BAD; or overexpressing BCL-2, and not overexpressing BCL-w and BAD; or overexpressing BCL-2, and not overexpressing BCL-xL, BCL-w, and BAD.

Because it has now been found that overexpression of BCL-xL reduces the effectiveness of BET bromodomain inhibitors to treat cancers, the instant disclosure is based on assessing whether the subject's cancer is overexpressing BCL-xL. Subjects whose cancer is not overexpressing BCL-xL can be selected and administered one or more BET bromodomain inhibitors. As used herein, the term “BET bromodomain inhibitor” is defined as a compound that binds to and/or inhibits the target bromodomain-containing protein (such as a BET protein, e.g., BRD2, BRD3, BRD4, and/or BRDT) with measurable affinity. In certain embodiments, an inhibitor has an IC₅₀ and/or binding constant of less than about 50 μM, less than about 1 μM, less than about 500 nM, less than about 100 nM, or less than about 10 nM.

It will be understood that “BCL2L1” refers to the gene encoding for the protein BCL-xL. As used herein, over- or under-expression of the gene BCL2L1 as a marker for use of the compounds described herein, is meant to be used interchangeably with over- or under-expression of BCL-xL since over- or under-expression of the underlying gene (BCL2L1) correlates with protein expression (BCL-xL).

Similarly, it will be understood that “BCL2L2” refers to the gene encoding for the protein BCL-w. As used herein, over- or under-expression of the gene BCL2L2 as a marker for use of the compounds described herein, is meant to be used interchangeably with over- or under-expression of BCL-w since over- or under-expression of the underlying gene (BCL2L2) correlates with protein expression (BCL-w).

It will be further understood that “BCL2” refers to the gene encoding for the protein BCL-2. As used herein, over- or under-expression of the gene BCL2 as a marker for use of the compounds described herein, is meant to be used interchangeably with over- or under-expression of BCL-2 since over- or under-expression of the underlying gene (BCL2) correlates with protein expression (BCL-2).

Because it has also now been found that overexpression of BCL-w reduces the effectiveness of BET bromodomain inhibitors to treat cancers, the instant disclosure is based on assessing whether the subject's cancer is overexpressing BCL-w. Subjects whose cancer is not overexpressing BCL-w can be selected and administered one or more BET bromodomain inhibitors. In certain embodiments, an inhibitor has an IC₅₀ and/or binding constant of less than about 50 μM, less than about 1 μM, less than about 500 nM, less than about 100 nM, or less than about 10 nM.

Because it has also now been found that overexpression of BAD reduces the effectiveness of BET bromodomain inhibitors to treat cancers, the instant disclosure is based on assessing whether the subject's cancer is overexpressing BAD. Subjects whose cancer is not overexpressing BAD can be selected and administered one or more BET bromodomain inhibitors. In certain embodiments, an inhibitor has an IC₅₀ and/or binding constant of less than about 50 μM, less than about 1 μM, less than about 500 nM, less than about 100 nM, or less than about 10 nM.

Because it has also now been found that overexpression of BCL-2 increases the effectiveness of BET bromodomain inhibitors to treat cancers, the instant disclosure is based on assessing whether the subject's cancer is overexpressing BCL-2. Subjects whose cancer is overexpressing BCL-2 can be selected and administered one or more BET bromodomain inhibitors. In certain embodiments, an inhibitor has an IC₅₀ and/or binding constant of less than about 50 μM, less than about 1 μM, less than about 500 nM, less than about 100 nM, or less than about 10 nM.

Additionally, combinations of markers can also be used. For example, the instant disclosure is also based on assessing whether the subject's cancer is overexpressing two or more of BCL-xL, BCL-2, BCL-w, and BAD. Subjects whose cancer is overexpressing BCL-2 and not overexpressing at least one of BCL-xL, BCL-w, and BAD can be selected and administered one or more BET bromodomain inhibitors. In certain embodiments, an inhibitor has an IC₅₀ and/or binding constant of less than about 50 μM, less than about 1 μM, less than about 500 nM, less than about 100 nM, or less than about 10 nM.

Methods for assessing overexpression of BCL-xL, BCL-2, BCL-w, and BAD in cancers can be achieved by procedures known those practicing in such fields. For example, tissue samples or biopsies may be taken from the tumor and the tissue sample or biopsy from the tumor may then be assessed according to routine procedures in the art filed for analyzing the expression of BCL-xL, BCL-2, BCL-w, and BAD in cancers. Such procedures include, e.g., qPCR, RNA in situ hybridization (RNA-ISH), whole genome expression profiling by array-based methods (e.g. Affymetrix), RNA sequencing (whole transcriptome shotgun sequencing), Northern blotting, Western Blotting, ELISA, Nanostring technology, Fluidigm, digital droplet PCR, Quantigene, Meso Scale Discovery electrochemiluminescence detection, and in situ hybridization (IHC).

Also, provided herein, according to one aspect, are packaged compositions comprising, a therapeutically effective amount of a BET inhibitor; and a pharmaceutically acceptable carrier or diluent, wherein the composition is formulated for treating a subject suffering from a cancer that is overexpressing one or more of BCL-xL, BCL-w, and BAD, and further optionally not overexpressing BCL-2, and wherein the composition is packaged with instructions to treat a subject suffering from a cancer that is overexpressing one or more of BCL-xL, BCL-w, and BAD, and further optionally not overexpressing BCL-2.

The BET bromodomain inhibitors described in the methods herein may be selected from any small molecule that targets BET proteins. Such inhibitors may include e.g., diazepines and derivatives, quinoline derivatives, dihydroquinazolinones, quinazolinones, dimethylisoxazoles, sulfonamides, thiazolidinones, thienodiazepenes, benzodiazepines and related analogues, triazolodiazepines and analogues, etc., and those referenced in Gamier et al., Expert Opin. Ther. Patents (2014) 24(2), WO 2009/084693, WO 1998/011111, WO 2006/129623, WO 2011/143669, WO 2011/143660, WO 2011/143651, WO 2013/030150, WO 2011/054845, WO 2011/054844, WO 2011/161031, WO 2012/075383, WO 2012/151512, WO 2013/027168, WO 2011/054848, WO 2011/054846, WO 2011/054843, WO 2013/024104, WO 2008/092231, WO 2009/158404, WO 2010/123975, WO 2012/174487, WO 2013/097601, WO 2013/033268, and WO 2012/116170, each of which are incorporated herein by reference.

In one aspect, the BET bromodomain inhibitors in the methods described herein are selected from:

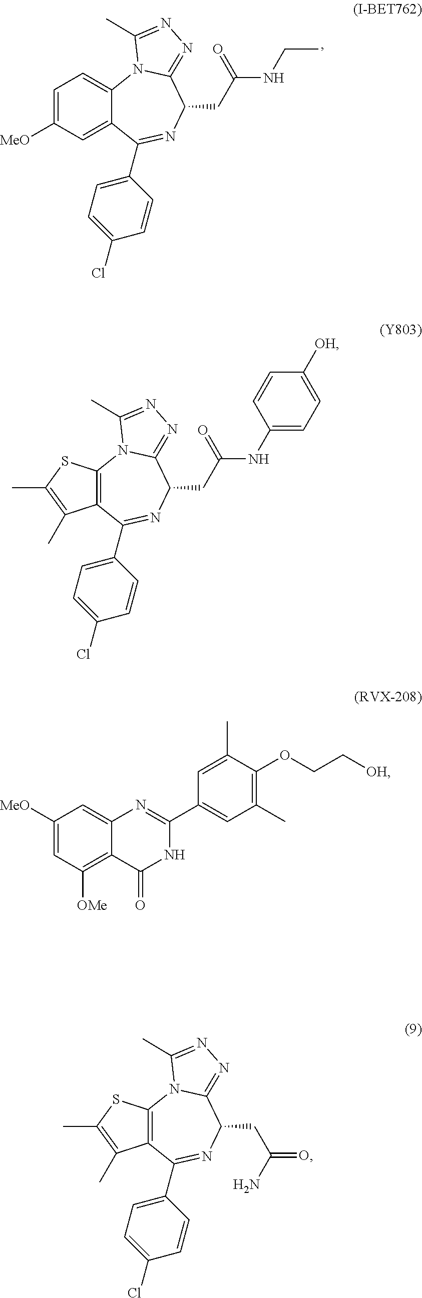

TEN-0110 (Tensha) ABBV-075 (Abbvie), BAY1238097 (Bayer), compounds described in US 2014/0275030, or pharmaceutically acceptable salts thereof, and compounds provided in 2012/075383, such as e.g., those represented by the following structural formula:

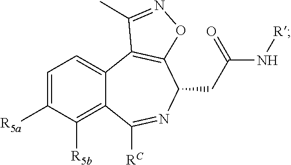

or pharmaceutically acceptable salts thereof, wherein: R₅ₐ is selected from hydrogen, halo, and alkoxy; R_{5b} is selected from hydrogen, halo, and alkyl; R^{C} is selected from phenyl, heteroaryl, and saturated heterocyclyl, wherein the group represented by R^{C} is optionally substituted with 1 to 2 substituents independently selected from halo, —CN, alkyl, alkoxy, haloalkoxy, haloalkyl, and carbamyl; and R′ is selected from hydrogen, alkyl, and alkoxyalkyl.

In another aspect, the BET bromodomain inhibitors in the methods described herein are

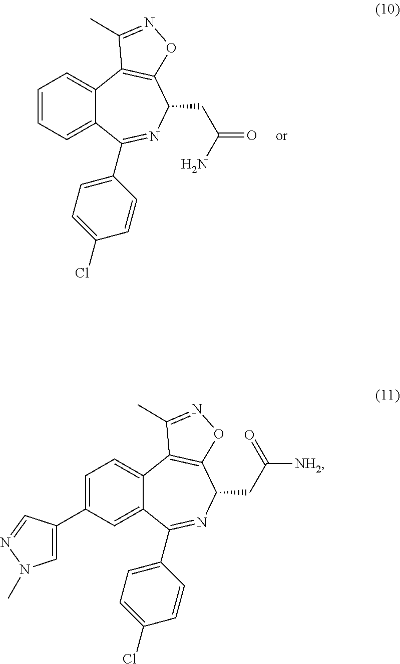 [SMILES: Cc1noc2c1-c1ccccc1C(c1ccc(Cl)cc1)=N[C@H]2CC(N)=O ; Cc1noc2c1-c1ccc(-c3cnn(C)c3)cc1C(c1ccc(Cl)cc1)=N[C@H]2CC(N)=O]

or a pharmaceutically acceptable salt thereof. The synthesis of these compounds can be found in WO 2012/075383 and WO 2013/184876.

For subjects whose cancers are found to overexpress BCL-xL, one may continue to treat with a BET bromodomain inhibitor, provided, however, that the expression or activity of BCL-xL is reduced. The activity of BCL-xL can be reduced, e.g., by administering to the subject an effective amount of a BCL-xL inhibitor together with an effective amount of a BET bromodomain inhibitor.

Alternatively, for subjects whose cancers are found to be overexpressing BCL-xL, one may elect treating the subject with an effective amount of a cancer therapy other than the administration of a BET bromodomain inhibitor. Such methods include e.g., surgery, radiation, immunotherapy, anti-cancer drugs, and the like.

Alternatively, subjects whose cancers are found to be overexpressing two or more of BCL-xL, BCL-w, and BAD, one may elect treating the subject with an effective amount of a cancer therapy other than the administration of a BET bromodomain inhibitor. Such methods include e.g., surgery, radiation, immunotherapy, anti-cancer drugs, and the like.

Alternatively, subjects whose cancers are found to not be overexpressing BCL-2, one may elect treating the subject with an effective amount of a cancer therapy other than the administration of a BET bromodomain inhibitor. Such methods include e.g., surgery, radiation, immunotherapy, anti-cancer drugs, and the like.

Alternatively, subjects whose cancers are found to not be overexpressing BCL-2 and overexpressing one or more of BCL-xL, BCL-w, and BAD, one may elect treating the subject with an effective amount of a cancer therapy other than the administration of a BET bromodomain inhibitor. Such methods include e.g., surgery, radiation, immunotherapy, anti-cancer drugs, and the like.

As used herein, the term “overexpressing” or when used in relation to BCL-xL, BCL-2, BCL-w, and BAD as in “overexpressing BCL-xL”; “overexpressing BCL-2”; “overexpressing BCL-w”; and “overexpressing BAD” means excessive expression of BCL-xL, BCL-2, BCL-w, and BAD respectively such that the expression of BCL-xL, BCL-2, BCL-w, and BAD respectively correlate with phenotypic resistance to BET bromodomain inhibition. When used in connection with “BCL-xL”; “BCL-2”; “BCL-w”; and “BAD” the terms “overexpressing” and “overexpression” are used interchangeably. For example, an increase in the level of one or more of BCL-xL, BCL-2, BCL-w, and BAD in a cell relative to the level in the same cell or closely related non-malignant cell under normal physiological conditions is a representation of overexpression as defined herein.

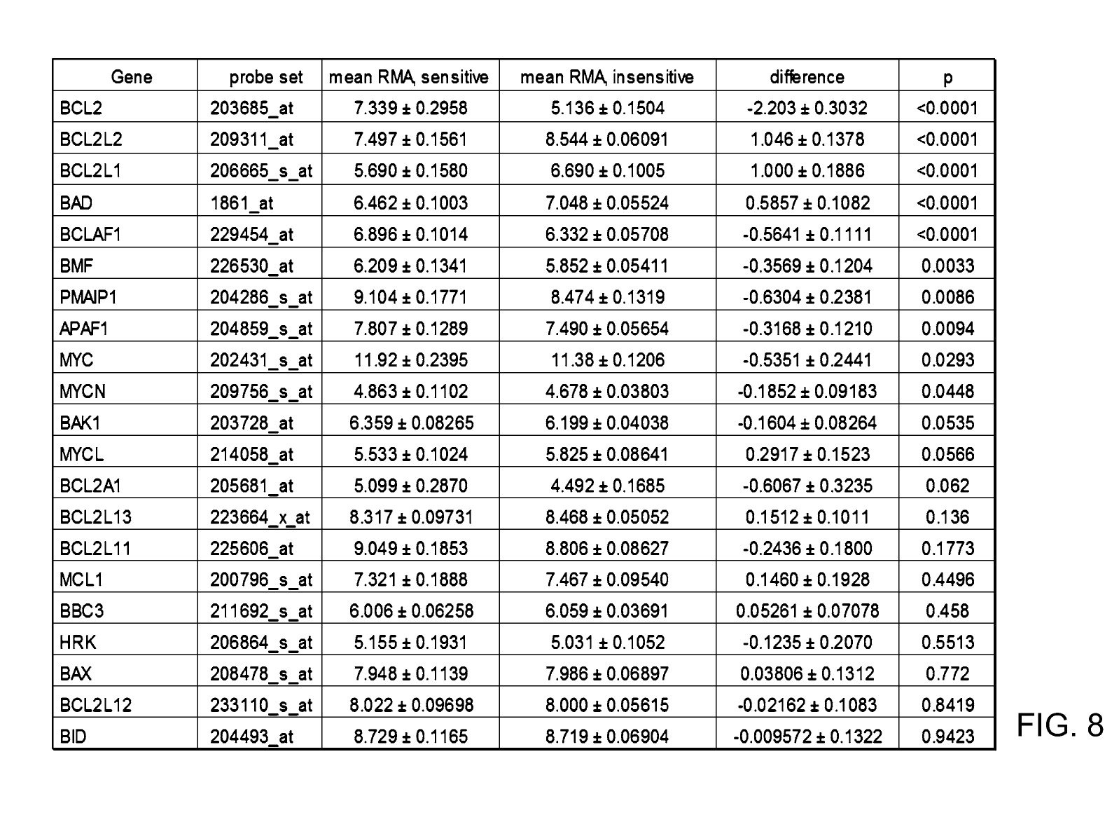

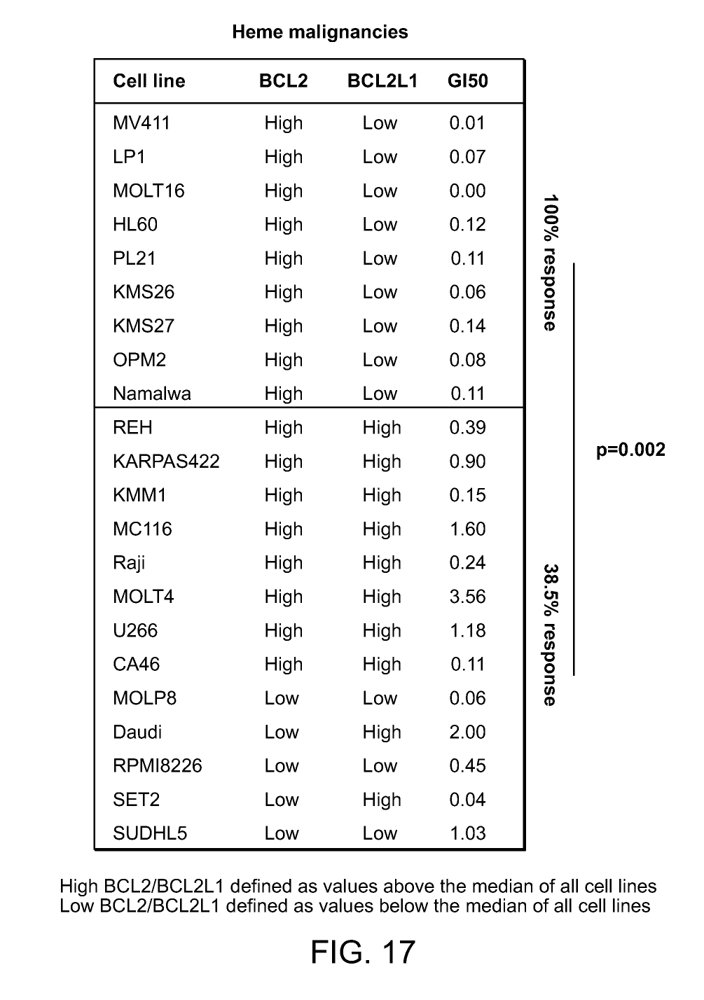

A subject overexpressing BCL-xL, BCL-2, BCL-w, and BAD may further be defined in terms of “high” expression or “low” expression. “High expression” means phenotypic resistance to BET bromodomain inhibition, wherein expression of BCL-xL is defined by a Robust Multichip Average (RMA) score above 5.5, above 6.0, above 6.5, or above 7.0 as obtained from the gene expression data used to derive the chart shown in FIG. 8. More specifically, the expression of BCL-xL was obtained from the CCLE expression profiling data published in Barrentina J, et al. (2012) Nature 483, 603-607, which is also described in Method A below. “High” expression of BCL-xL can also be defined by a qPCR score of above 0.5, above 0.7, about 1.0, or above 1.2 as obtained by the methods according to FIG. 17, or equivalent expression to at least 50^{th} percentile or higher (e.g., at least 55^{th}, 60^{th}, 65^{th}, or 67^{th} percentile or higher) expression of BCL-xL across all of the cell lines tested in FIG. 8s or FIG. 17. “Low expression” means no phenotypic resistance to BET bromodomain inhibition, wherein expression of BCL-xL is defined by a RMA score of less than 5.5, 6.0, 6.5, or 7.0 and/or a qPCR score of less than 0.5, 0.7, 1.0, or 1.2. Not overexpressing means low expression of BCL-xL, BCL-2, BCL-w, and BAD, or other than high expression.

Alternatively, “High expression” of BCL-2, BCL-xL, BCL-w, or BAD can be defined as a level of expression of mRNA or protein in a sample of a tumor that is 1.5 fold above the level in a tissue-matched non-malignant cell from the same patient.

In another alternative, “high expression” can be defined as having greater than 2 copies of the genes for BCL-2, BCL-xL, BCL-w, or BAD in a tumor sample, as measured by techniques such as such as fluorescent in situ hybridization, comparative genomic hybridization, array comparative genomic hybridization, and single nucleotide polymorphism array technologies.

In another alternative, “high expression” of BCL-2, BCL-xL, BCL-w, or BAD can be defined as a tumor sample containing genomic translocations involving these genes that are known to promote high expression (e.g. the t(14; 18) translocation in diffuse large B-cell lymphoma (Souers A. J. et al. (2013) “ABT-199, a potent and selective BCL-2 inhibitor, achieves antitumor activity while sparing platelets” Nature Medicine 19: 202-208). Such translocations can be detected by techniques such as fluorescence in situ hybridization or genomic sequencing.

In another alternative, “high expression” can be defined as a tumor sample in which >10% of cells express BCL-2, BCL-xL, BCL-w, or BAD as determined by immunohistochemistry techniques.

In another alternative, “high expression” means an amount of cellular BCL-2, BCL-xL, BCL2L1, or BAD above 0.1 ng per ug of total cellular protein, or above 0.5 ng per ug of total cellular protein, or above 1 ng per ug of total cellular protein as described in Souers, A. J., et al. (2013) “ABT-199, a potent and selective BCL-2 inhibitor, achieves antitumor activity while sparing platelets.” Nature Medicine 19: 202-210.

Alternatively, “high expression” means expression that falls within at least 50^{th} percentile or higher (e.g., at least 55^{th}, 60^{th}, 65^{th}, or 67^{th} percentile or higher) of BCL-xL, BCL-2, BCL-w, or BAD expression obtained from tumor samples obtained from a population of individuals with the cancer in terms of the level of BCL-xL, BCL-2, BCL-w, and BAD expressions. Population is defined as being at least 30 individuals, at least 50 individuals, at least 100 individuals, at least 1,000 individuals, at least 2,500 individuals, or at least 10,000 or more individuals, and level of expression being determined by an appropriate means, including e.g., qPCR, RNA in situ hybridization (RNA-ISH), whole genome expression profiling by array-based methods (e.g. Affymetrix), RNA sequencing (whole transcriptome shotgun sequencing), Northern blotting, Western Blotting, ELISA, Nanostring technology, Fluidigm, digital droplet PCR, Quantigene, Meso Scale Discovery electrochemiluminescence detection, in situ hybridization (IHC). Methods for quantification of BCL-2, BCL-xL, BCL-w, and BAD expression levels can performed e.g., using expression profiling (Affymetrix) or by Expression profiling (qPCR).

Each of the expression values defined above, either alone or in combination with one another may be associated with any of the methods described herein. For example, in one or more of the methods described herein, overexpression may comprises a level of expression of mRNA or protein in a sample of a tumor that is 1.5 fold above the level in a tissue-matched non-malignant cell from the same patient; and/or comprise greater than 2 copies of the genes for BCL-2, BCL-xL, BCL-w, or BAD in a tumor sample; and/or comprise a tumor sample containing genomic translocations involving these genes that are known to promote high expression; and/or comprise a tumor sample in which >10% of cells express BCL-2, BCL-xL, BCL-w, or BAD as determined by immunohistochemistry techniques; and/or comprise an amount of cellular BCL-2, BCL-xL, BCL2L1, or BAD above 0.1 ng per ug of total cellular protein.

A. Expression Profiling (Affymetrix): “Low” Expression of BCL-xL was Classified with an RMA Score of Below 5.6. See e.g., FIG. 8.

Adapted from: Barrentina J, et al. (2012) Nature 483, 603-607. mRNA expression data was obtained using Affymetrix Human Genome U133 Plus 2.0 arrays according to the manufacturer's instructions. Array preparation and scanning was performed by the Genomics Analysis Platform at the Broad Institute. Genecentric expression values were obtained using updated Affymetrix probe set definition files (CDF files) from Brainarray (Dai, M. et al. Evolving gene/transcript definitions significantly alter the interpretation of GeneChip data. Nucleic Acids Res 33, e175, (2005)); and background correction was accomplished using RMA (Robust Multichip Average) (Irizarry, R. A. et al. Exploration, normalization, and summaries of high density oligonucleotide array probe level data. Biostatistics 4, 249-264, (2003)) and quantile normalization (Bolstad, B. M., Irizarry, R. A., Astrand, M. & Speed, T. P. A comparison of normalization methods for high density oligonucleotide array data based on variance and bias. Bioinformatics 19, 185-193, (2003)). Quality assessment was performed to identify low performing microarrays, using the R package affyPLM (Brettschneider, J., Collin, F. o., Bolstad, B. M. & Speed, T. P. Quality Assessment for Short Oligonucleotide Microarray Data. Technometrics 50, 241-264, (2008)). Outliers in the distribution of NUSE, RLE, background signal and percentage of “present” genes were flagged to be re-processed. In addition, all microarray pseudoimages were checked visually. RMA values for each cell line were ranked, and BCL-xL expression was classified as “low” if the value was in the bottom 33% of all cell lines.

B. Expression Profiling (qPCR): “Low” Expression of BCL-xL was Classified as a Score of 0.5 or Less in the Cell Lines Tested, and “High” Expression of BCL-2 was Classified as a Score of 0.04 or Greater in the Cell Lines Tested.

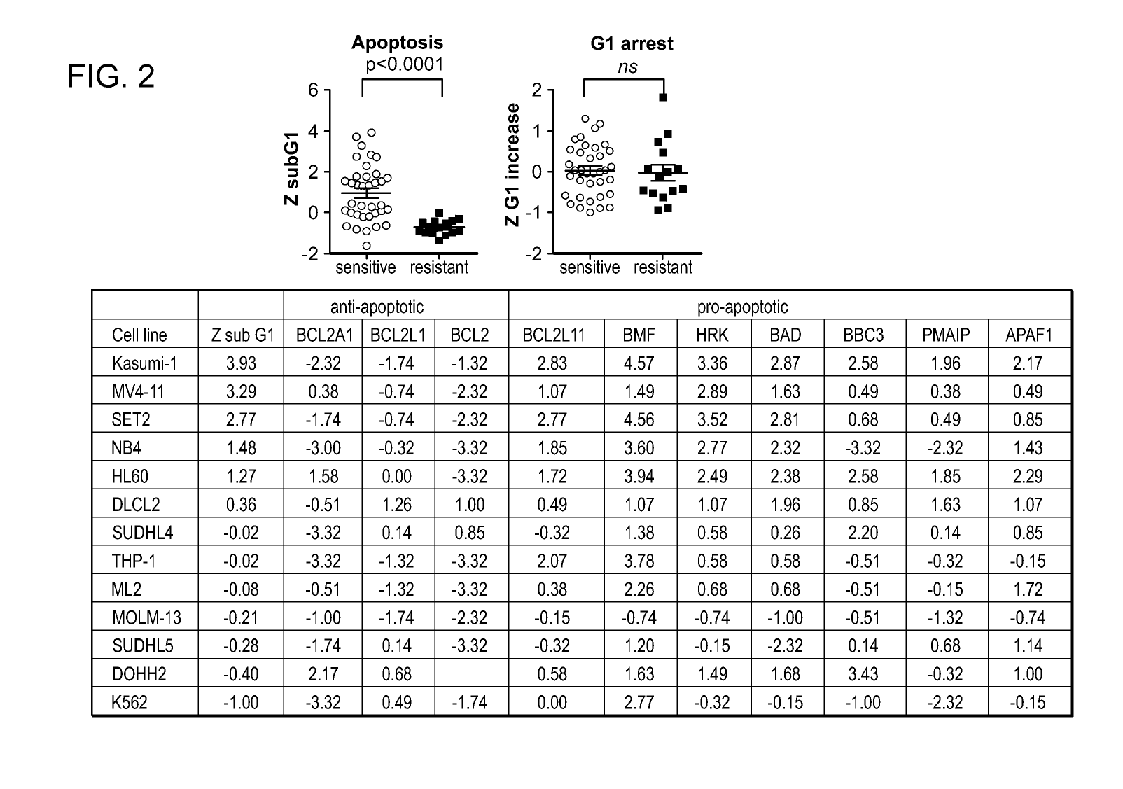

Standard qPCR techniques were used to determine the relative expression of BCL-xL in 22 cell lines reported in FIG. 17. Ct values were normalized using a housekeeping gene (PPIB) and ratios were calculated using the normalized expression of BCL-xL and BCL-2 in the Karpas-422 cell line (value set to 1.0). “High” expression of BCL-xL or BCL-2 was defined as a ratio of greater than 0.5 or 0.04, respectively. “Low” expression of BCL-xL or BCL-2 was defined as a ratio of less than 0.5 or 0.04, respectively. The composite GI50 values of the respective cell lines are shown. GI50 values were determined as described in FIG. 2. Sensitive cell lines were classified as having a GI50, of less than 0.25 μM. As shown in FIG. 17, “high” expression of BCL-2 and “low” expression of BCL-xL correlates with sensitivity to BET bromodomain inhibition.

In one aspect, the BCL-xL inhibitors in the methods described herein are selected from any small molecule inhibitors that broadly target BCL-2 family members. Such inhibitors may include e.g., non-peptide small molecule inhibitors that broadly target BCL-2 family members (including BCL-xL), as well as those that selectively target BCL-xL. In another aspect, peptide-based or peptidomimetic BCL-xL inhibitors are also intended in the methods described herein. In a further aspect, therapies that result in a reduction of protein expression including RNAi, antisense oligonucleotides, and genome editing are also contemplated in the instant methods. Non-limiting examples of BCL-xL inhibitors based on the methods described herein can be found in e.g., Bajwa et al., Expert Opin Ther Pat 2012 January 22(1): 37-55, WO 2004/058804, WO 2006/000034, WO 2005/044839, U.S. Pat. No. 7,723,469, WO 2002/097053, U.S. Pat. No. 7,432,304, WO 2005/069771, WO 2005/094804, U.S. Pat. No. 7,342,046, U.S. Pat. No. 7,432,300, WO 2008/150506, WO 2009/045410, WO 2006/050447, WO 2009/052443, U.S. Pat. No. 8,039,668, WO 2010/120943, WO 2006/023778, WO 2004/106328, WO 2005/117908, U.S. Pat. No. 7,425,553, WO 2006/069441, US 20070072860, U.S. Pat. No. 7,642,260, U.S. Pat. No. 7,973,161, WO 2002/024636, WO 2005/049593, WO 2005/049594, U.S. Pat. No. 7,767,684, U.S. Pat. No. 7,906,505, WO 2006/127364, U.S. Pat. No. 7,777,076, WO 2005/117543, U.S. Pat. No. 7,585,858, WO 2009/155386, WO 2010/083442, WO 2010/065865, WO 2008/130970, U.S. Pat. No. 7,981,888, WO 2008/131000, WO 2007/008627, U.S. Pat. No. 7,750,004, U.S. Pat. No. 7,989,656, WO 2002/060887, U.S. Pat. No. 6,660,871, WO 2001/14365, U.S. Pat. No. 7,241,804, WO 2008/060569, U.S. Pat. No. 7,842,815, U.S. Pat. No. 7,851,637, WO 2006/002474, and U.S. Pat. No. 7,956,216, each of which are incorporated herein by reference. In a further aspect, the BCL-xL inhibitors in the methods described herein are selected from

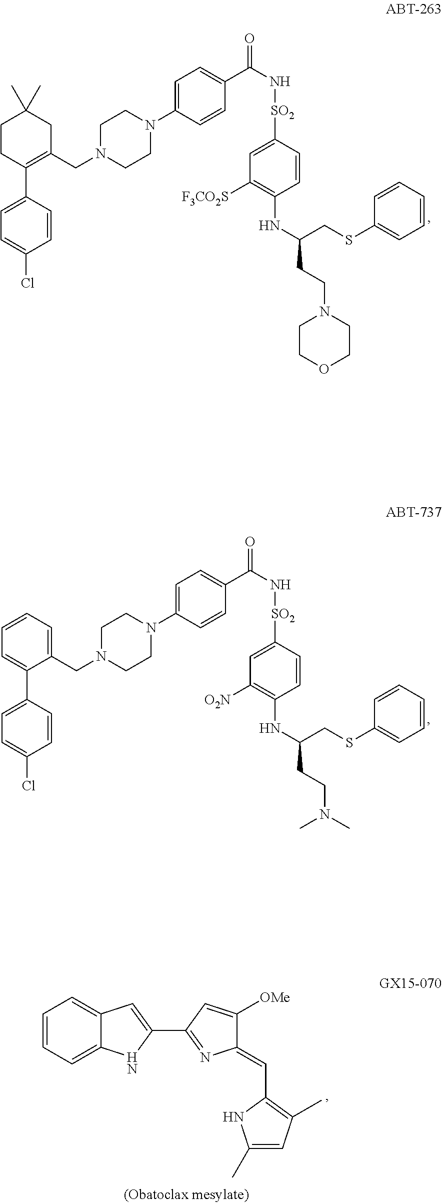

and (−)-1,1′,6,6′,7,7′-Hexahydroxy-3,3′-dimethyl-5,5′-bis(1-methylethyl)-[2,2′-binaphthalene]-8,8′-dicarboxaldehyde AT-101 (gossypol), or pharmaceutically acceptable salts thereof.

It should be understood administration routes, specific dosages, and treatment regimens using the compounds of the methods described herein will also depend upon a variety of factors, including age, body weight, general health, sex, diet, time of administration, rate of excretion, drug combination, the judgment of the treating physician, and the severity of the particular disease being treated.

In one aspect, the cancers treatable by the disclosed methods include midline carcinomas, neuroblastomas, cancers of the lung (large and small), breast, prostate, thyroid, tongue, mouth, pharynx, esophagus, stomach, intestine, colon, rectum, anal canal, liver, bile duct, pancreas, larynx, bone, joints, soft tissue, skin, uterine, ovary, vulva, vagina, testis, bladder, kidney, ureter, eye, and brain cancers. Alternatively, the cancers described in the present methods are selected from hematological malignancies. Such malignancies include e.g., Non-Hodgkin's lymphoma, Hodgkin's lymphoma, acute lymphocytic leukemia, chronic lymphocytic leukemia, acute myeloid leukemia, chronic myeloid leukemia, myelodysplastic syndromes, myeloproliferative neoplasms, or multiple myeloma.

In another aspect, the cancers treatable by the disclosed methods include midline carcinomas, neuroblastomas, cancers of the lung (large and small), breast, prostate, thyroid, tongue, mouth, pharynx, esophagus, stomach, intestine, colon, rectum, anal canal, liver, bile duct, pancreas, larynx, bone, joints, soft tissue, skin, uterine, ovary, vulva, vagina, testis, bladder, kidney, ureter, eye, and brain cancers. Alternatively, the cancers described in the present methods are selected from hematological malignancies. Such malignancies include e.g., Non-Hodgkin's lymphoma, Hodgkin's lymphoma, acute lymphocytic leukemia, chronic lymphocytic leukemia, acute myeloid leukemia, chronic myeloid leukemia, myelodysplastic syndromes, myeloproliferative neoplasms, myelodysplastic/myeloproliferative neoplasms, or multiple myeloma.

As used herein, the terms “treatment,” “treat,” and “treating” refer to reversing, alleviating, or inhibiting the progress of a cancer, or one or more symptoms thereof, as described herein.

The term “subject” as used herein refers to a mammal. A subject therefore refers to, for example, dogs, cats, horses, cows, pigs, guinea pigs, and the like. Preferably the subject is a human. When the subject is a human, the subject may be either a patient or a healthy human.

As used herein, the term “pharmaceutically acceptable salt” refers to those salts of the compounds described herein that are within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66: 1-19 (1977).

### Correlation Between Apoptosis and Sensitivity to BET Inhibition

Approximately 60 hematological cancer cell lines were profiled, with representative numbers of leukemia, multiple myeloma/plasmacytoma, and lymphoma cell lines. Cells were grown in the presence of BET inhibitors, (9), (10), and (11), for 4 days. Viability was measured using a fluorescent dye (resazurin, Sigma) that measures mitochondrial fitness as a proxy for cell number. Raw fluorescence values at each compound concentration were normalized to the values in the absence of compound, and percentage growth was plotted to determine the concentration of compound at which 50% growth inhibition was achieved (GI50). Cells were then fixed and stained with propidium iodide for flow cytometric determination of cell cycle distribution. The percentage of cells with less than G0/G1 DNA content (% subG1) was used as a measurement of apoptosis. The percentage of viable cells (non-subG1 cells) with G0/G1 DNA content was measured, and the % GI for control cells was subtracted from the % GI at each compound concentration to calculate a percentage increase in G1.

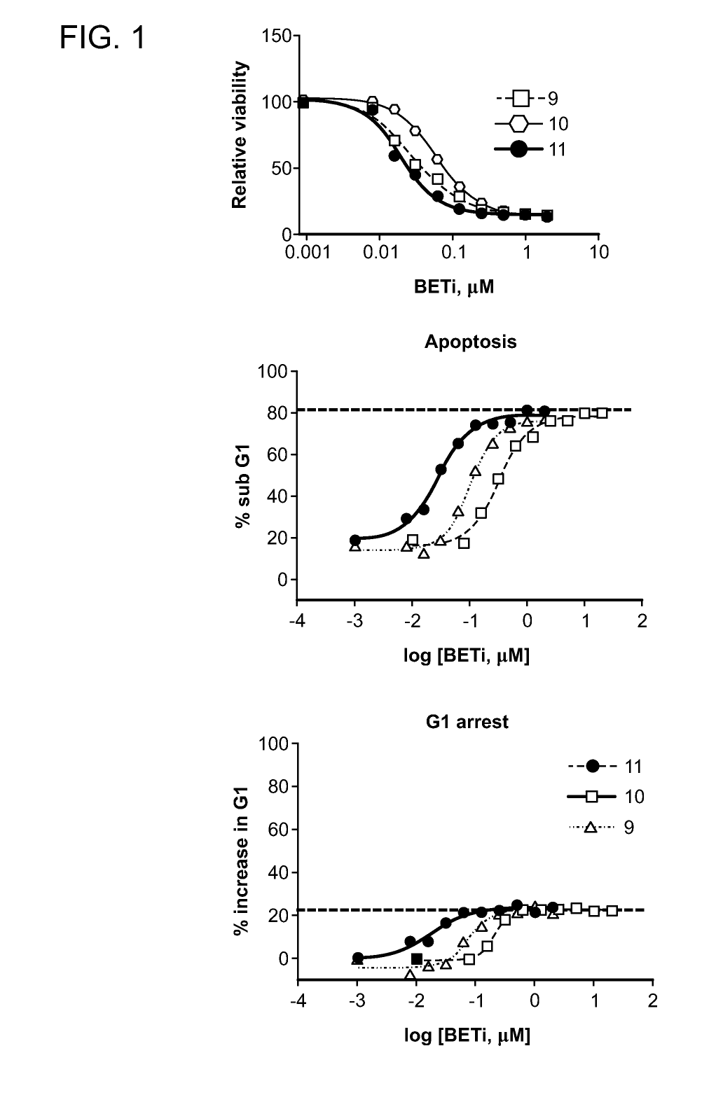

As shown in FIG. 1, the phenotypic response of cells to each inhibitor is very similar and correlates with the potency of the inhibitors. The top panel shows growth inhibition of the cell line KMS-28PE in response to 4 days of treatment with increasing concentrations of three BET inhibitors, (9), (10), and (11). The bottom panels show the dose dependent increase in the percentage of cells with sub-G1 DNA content (apoptosis) and G0/G1 DNA content (G1 arrest) in response to 4 days of treatment with the same BET inhibitors in the AMO-1 cell line. The dashed line demonstrates that the maximal response is equivalent for all three compounds.

A composite G150 score was determined by calculating the mean G150 across all replicates and all inhibitors, after first dividing the G150 for (10) by ten to adjust for the known difference in potency. Composite % sub-G1 and % G1 scores (Z subG1 and Z G1 increase) were determined by calculating the number of standard deviations in the increase in % sub-G1 or % G1 in each cell line relative to the median value of all of the cell lines. To eliminate effects of the compounds that might occur at non-physiologically relevant concentrations, composite % subG1 and % G1 increase were calculated at 0.25 μM (9) and (11) and 2.5 μM (10).

“Sensitive” cell lines were defined as those having a composite G150 score of less than 0.25 μM, and “resistant” cell lines were defined as those having a composite G150 score of greater than 0.25 μM. In FIG. 2, the Z subG1 (left panel) and Z G1 increase (right panel) were compared for sensitive and resistant cell lines. There is a significant increase in the Z subG1 for sensitive vs. resistant cells, whereas the Z G1 increase is not different between the two groups.

The modulation of mRNA expression of several genes in the apoptotic signaling network upon BET inhibition was monitored in 13 cell lines of hematopoetic origin using q-RTPCR. As shown in FIG. 2 (bottom panel), there is a correlation between the modulation of the expression of apoptotic factors (downregulation of pro-apoptotic factors and upregulation of anti-apoptotic factors) and the magnitude of the apoptotic response after 4 d treatment with BET inhibitors.

Taken together, these results indicated that an apoptotic response to BET inhibition (measured by phenotypic and transcriptional readouts) correlates with potent growth suppression, and that cell lines that do not undergo significant apoptosis in response to BET inhibitors tend to be more refractory. The observation that this relationship holds for BET inhibitors of multiple chemotypes suggests that this relationship is target-specific rather than compound-specific.

### Engineered BET Inhibitor Resistant Cells are Less Prone to BET Inhibitor-Induced Apoptosis and Overexpress BCL-xL

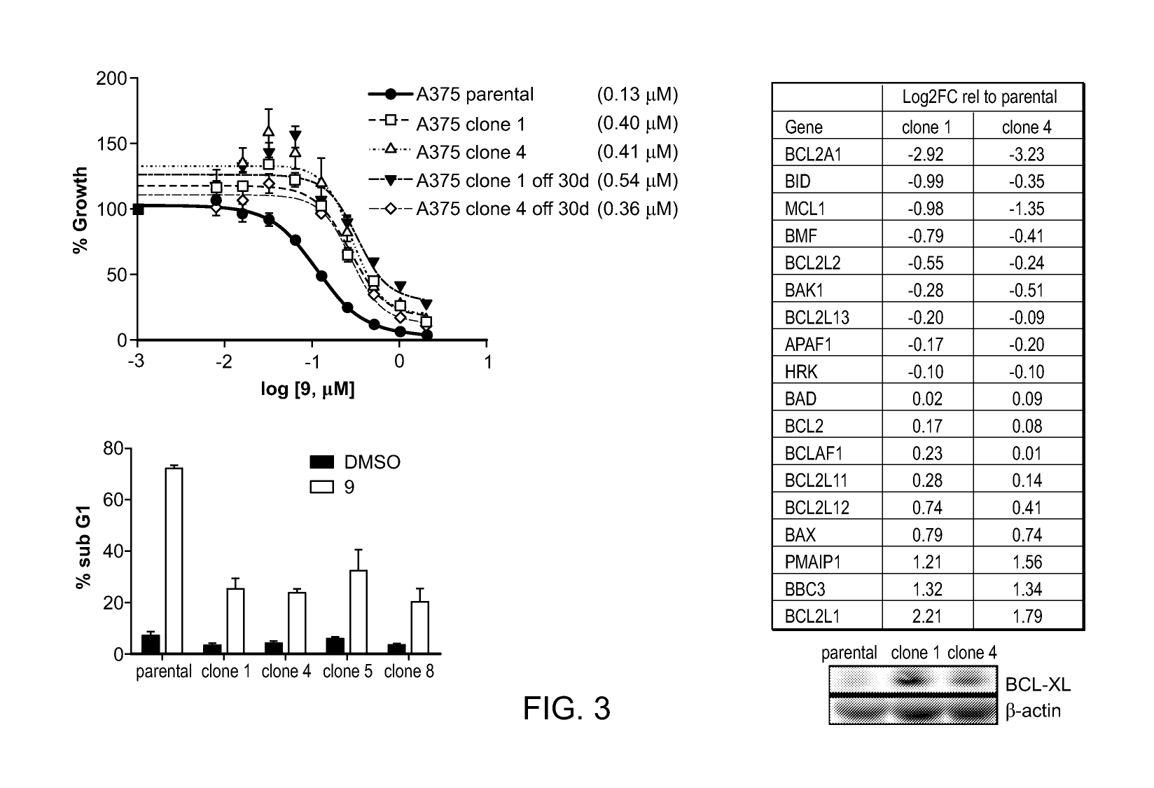

The human melanoma cell line A375 is phenotypically sensitive to BET inhibition, with a GI50 of 0.10 μM for (9) (FIG. 3, top panel). To understand possible mechanisms of acquired and de novo resistance to BET inhibition, A375 cells were grown in the presence of 1 μM of (9) for approximately 90 days. Viable clones were cultured in the continued presence of the compound, or were cultured in complete media without compound for 30 days. The majority of cells were eliminated, but several resistant clones grew out in the continued presence of the inhibitor. These resistant clones displayed a relatively modest shift in GI50 relative to parental cells after 11 days (FIG. 3, top panel), but notably the degree of apoptosis was significantly reduced in multiple clones (FIG. 3, middle panel).

To identify potential mechanisms for resistance, transcriptional profiling of parental A375 cells and 2 BET inhibitor resistant clones was carried out using RNA sequencing. When the expression level of various apoptosis-related genes was compared, it was observed that both BET inhibitor resistant clones had a significantly increased expression level of the anti-apoptotic BCL-2 family member BCL-xL (BCL2L1, FIG. 3, bottom panel). The increased mRNA expression level was associated with a corresponding increase in BCL-xL protein (FIG. 3, bottom panel).

### Protein Knockdown and Small Molecule Inhibition of BCL-xL Restore Sensitivity to BET Inhibition in BET Inhibitor Resistant A375 Cells

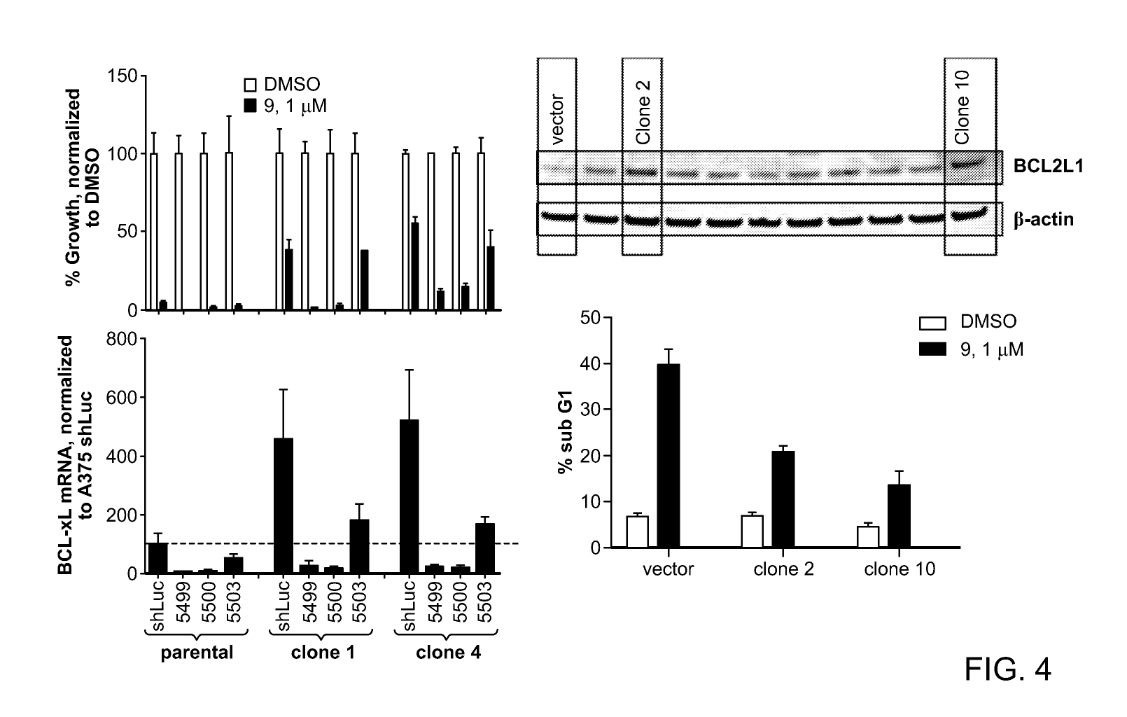

To address the functional relevance of increased BCL-xL expression in BET inhibitor resistant A375 cells, parental and resistant cells were treated with 1 μM of (9) for 11 days in the cells transduced with control luciferase shRNAs or shRNAs targeting BCL-xL. As shown in FIG. 4 (top 2 panels), knockdown of BCL-xL in BET inhibitor resistant A375 cells completely restored phenotypic sensitivity to BET inhibition. Importantly, the increased sensitivity was observed with two distinct targeting sequences, and the degree of knockdown of BCL-xL transcript and protein correlated with increased sensitivity to BET inhibition, suggesting that the effect is on target and directed against BCL-xL.

To further confirm the relevance of BCL-xL expression in defining the phenotypic response to BET inhibition, parental A375 cells were transduced with a BCL-xL expression vector BCL-xL expressing clones were isolated. As shown in FIG. 4 (bottom panels), overexpression of BCL-xL in parental A375 is sufficient to abrogate the apoptotic response to BET inhibition.

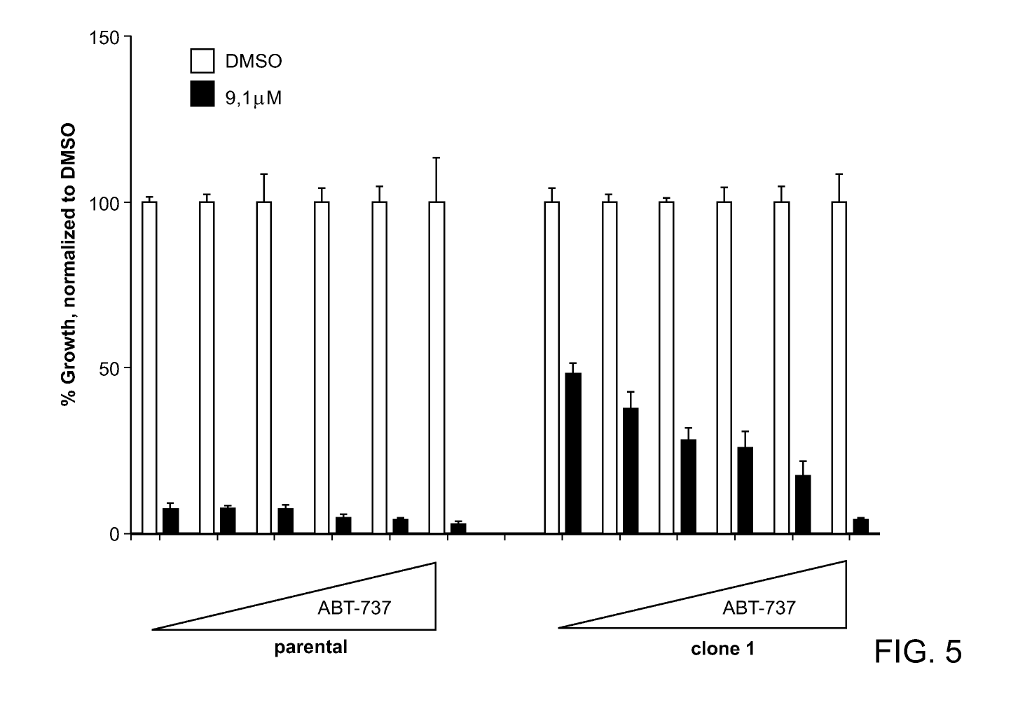

BCL-xL is targeted by several commercially available BCL-2 family small molecule inhibitors (Billard, C. et al. BH3 mimetics: status of the field and new developments. Mol Cancer Ther. 2013 September; 12(9):1691-700). To test whether pharmacological inhibition of BCL-xL would restore sensitivity to BET inhibition in BET inhibitor resistant A375 cells, parental and resistant cells were co-treated with 1 μM (9) or DMSO and increasing concentrations of the pan-BCL2 inhibitor ABT-737 for 11 days (Ottersdorf, T., et al. An inhibitor of Bcl-2 family proteins induces regression of solid tumours. Nature. 2005 Jun. 2; 435(7042):677-81). As shown in FIG. 5, exposure to ABT-737 restores sensitivity to (9) in a dose-dependent manner. While ABT-737 targets both BCL-2 and BCL-xL, BCL-2 was not detected in A375 cells, suggesting that the effect is due to engagement of BCL-xL.

### BETi-Tolerant NOMO-1 AML Cells Show Dysfunction in the Apoptotic Response

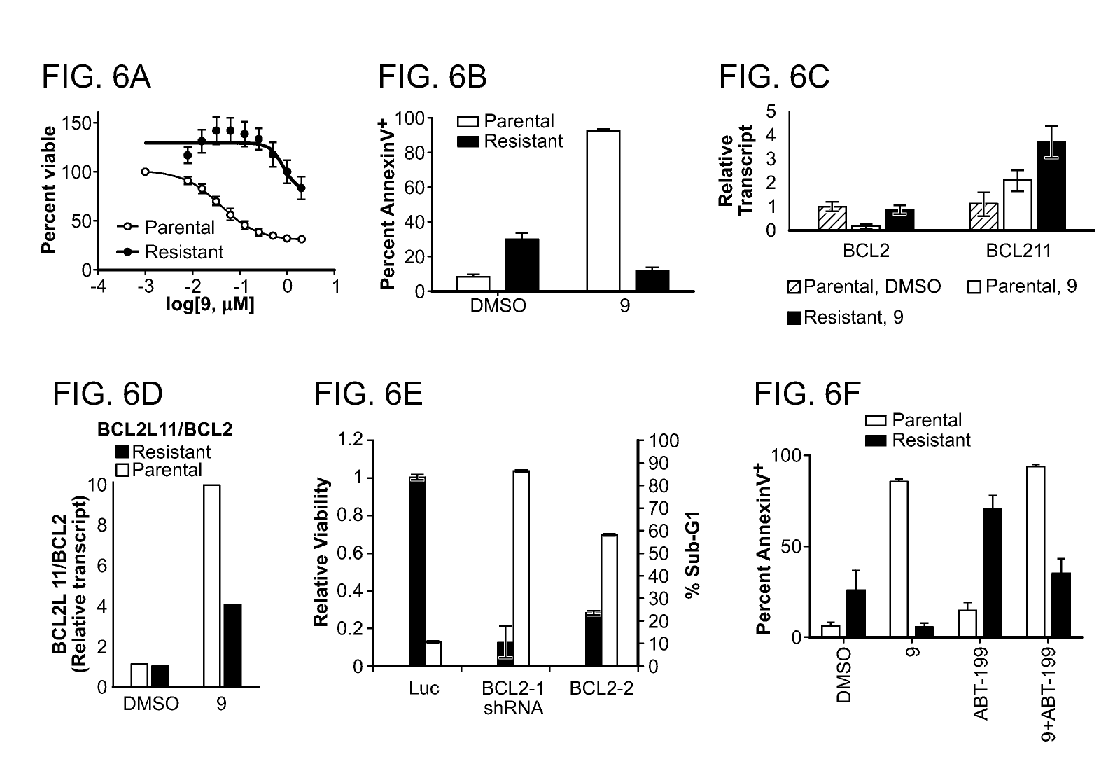

A BETi-resistant acute myeloid leukemia (AML) cell line was prepared. NOMO-1 cells were treated with increasing concentrations of (9), and when the population consisted of less than 80% live cells, viable cells were enriched by spinning over Ficoll-Paque (GE Healthcare). At the end of this procedure cells were able to proliferate in the presence of 1 μM (9), and were maintained at this dose, which is about 30-fold higher than their original GI50 (FIG. 6A). The ideal growth conditions for the BETi-tolerant cells appeared to be in the presence of ˜0.1 μM (9). While parental cells induced a robust apoptotic response in response to BETi, resistant cells grown in the presence of BETi only underwent apoptosis when the inhibitor was completely removed (FIG. 6B).

Given observations with BCL-xL in BETi-tolerant A375 cells, the expression of BCL2 family members in the tolerant NOMO-1 cells was examined. Unlike in the A375 system, parental NOMO-1 cells express BCL2, but have very low expression of BCL2L1. In response to BETi, parental NOMO-1 cells showed strong suppression of BCL2 transcript. BCL2 expression levels were restored in resistant cells in the presence of BETi (FIG. 6C).

Previous work has demonstrated that the robustness of the apoptotic response is governed by the balance between pro- and anti-apoptotic factors. (See e.g., Fernald and Kurokawa. Evading Apoptosis in Cancer. Trends Cell Biol. 2013 December; 23(12): 620-633.) Expression of the pro-apoptotic gene BCL2L11 (encoding BIM) was increased in parental cells upon treatment with BETi (FIG. 6C). The relative expression ratio of BCL2L11/BCL2 correlated with the apoptotic response in parental cells (FIG. 6D). In the resistant cells cultured in the presence of (9), high levels of BIM were maintained (FIG. 6C). This suggested that the maintenance of high BCL2 expression was necessary to counteract the pro-apoptotic function of BIM in these cells. This was consistent with the A375 model, in which several pro-apoptotic genes are upregulated in resistant clones (FIG. 3, e.g. PMAIP, BBC3), which is potentially counteracted by increased expression of BCL2L1. Also analogous to the A375 model, pharmacologic inhibition of BCL2 or reduction of BCL2 expression by RNAi resulted in a strong apoptotic response and a dramatic effect on viability in the resistant cells (FIGS. 6E-F). Thus, in this model of BETi resistance, modulation of the apoptotic signaling network via maintenance of BCL2 expression appeared to be one mechanism driving cell survival in the presence of BET inhibition.

Accordingly, it was found that in two different cell-of-origin models, closely related anti-apoptotic members of the BCL-2 family were upregulated/maintained upon achieving reduced phenotypic sensitivity to BET bromodomain inhibitors.

Expression Levels BCL2, BCL2L1, BCL2L2, and BAD are Correlated with Phenotypic Response to BET Inhibitors

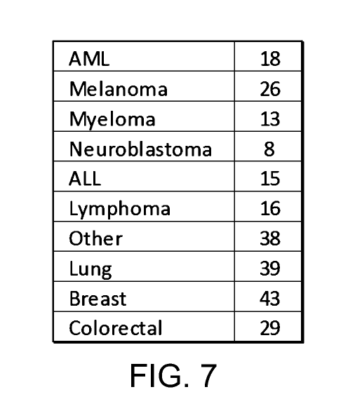

In order to identify predictive biomarkers for phenotypic response to BET inhibition, viability data was obtained for a panel of cell lines for which gene expression data are available. A panel of 245 cell lines of varying lineages (FIG. 7) was treated with the BET bromodomain inhibitor (9) for 3 or 4 d. Viability was assessed using either Cell Titer Glo (Promega) or resazurin (Sigma). GI50 values were determined as the concentration of inhibitor that caused viability to be reduced by 50% relative to the vehicle-treated control cells. Cell lines were defined as “sensitive” if GI50 values were at or below 0.25 μM, and as “insensitive” if GI50 values were above 0.25 μM.

Expression values of a panel of 21 genes (apoptotic factors and MYC family genes) were obtained from the Cancer Cell Line Encyclopedia (CCLE) database of RNA microarray expression data. Where multiple probe sets were available for a given gene, the probe set that hybridized to all transcript variants and gave the largest standard deviation across the panel of cell lines was chosen.

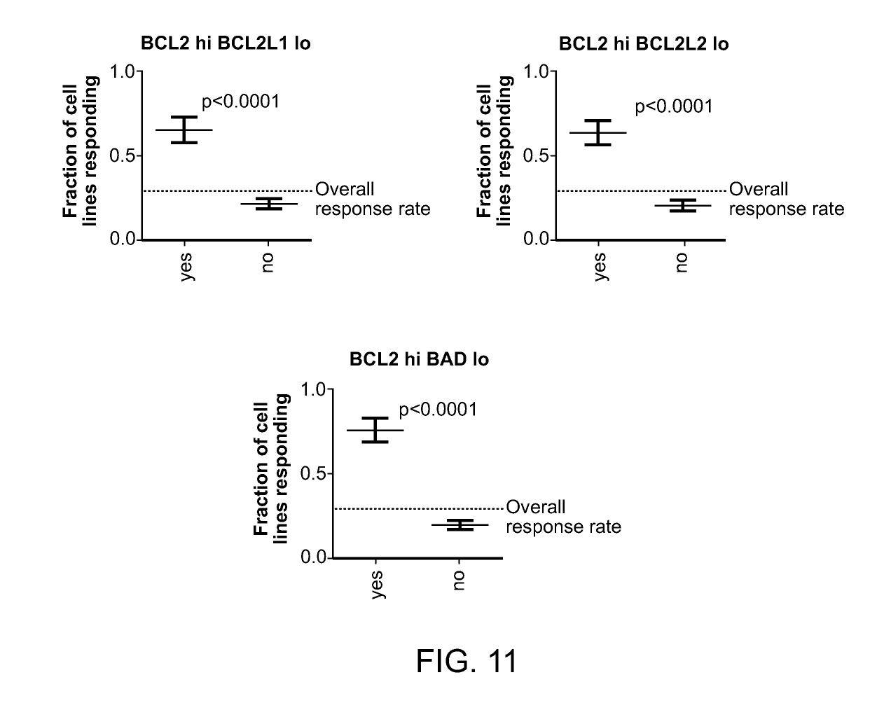

The mean and standard error in the expression value of each gene was determined for sensitive and insensitive cell lines, and significant differential expression between the two groups was determined as a p-value of less than 0.05 in an unpaired t-test. As shown in FIG. 8, multiple genes were differentially expressed between the two groups, with five genes (BCL2, BCL2L2, BCL2L1, BAD, and BCL2AF1) showing differential expression at p<0.0001 (FIG. 11). Notably, changes in the expression level of BCL2L1 and BCL2 were observed in models of acquired resistance to BET inhibition described above, suggesting that changes in the expression of apoptotic factors can play a role in both acquired and de novo resistance to BET inhibition.

### Expression of BCL2, BCL2L1, BCL2L2, and BAD Significantly Predict In Vitro Response to BET Inhibitors

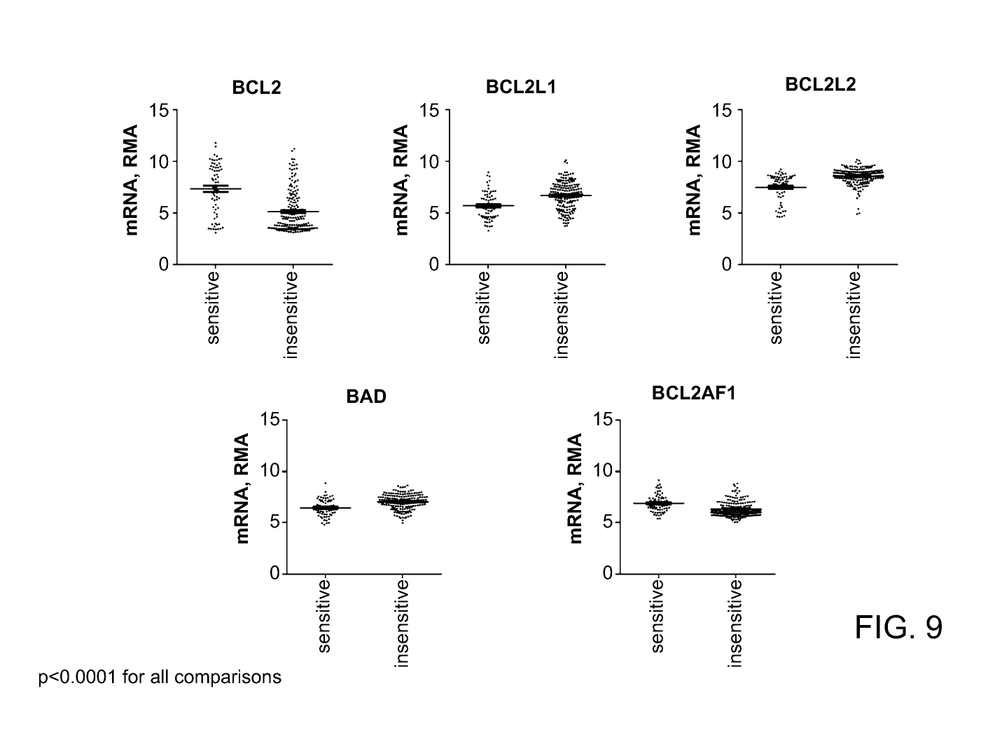

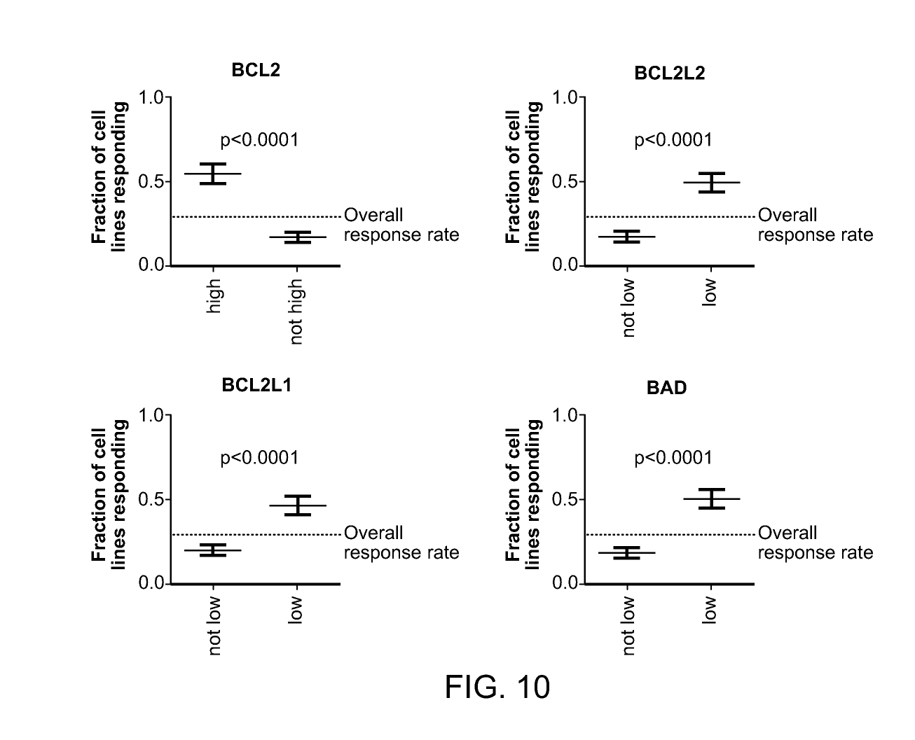

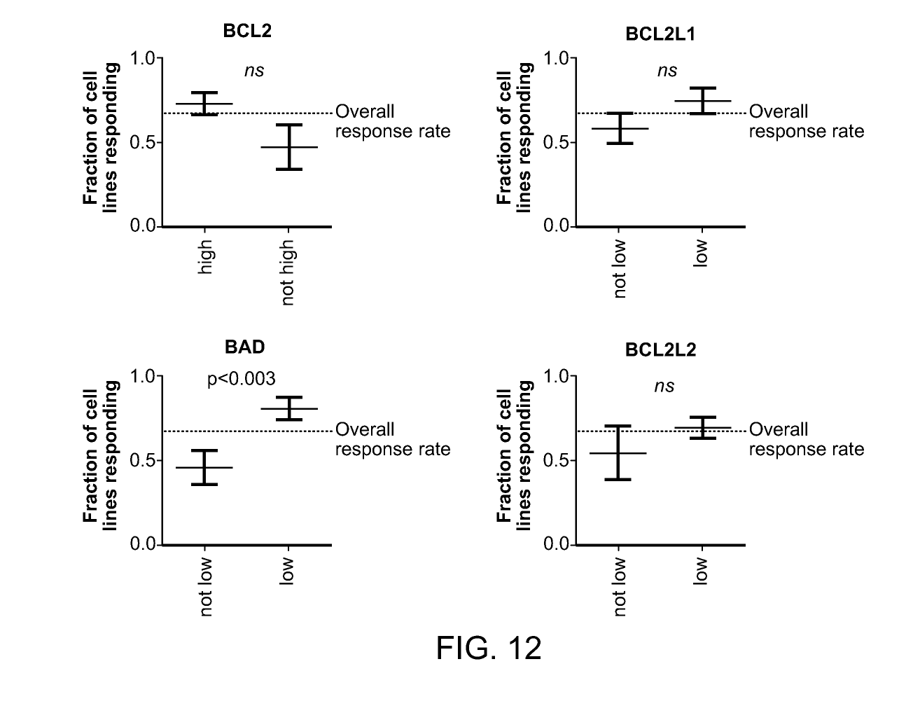

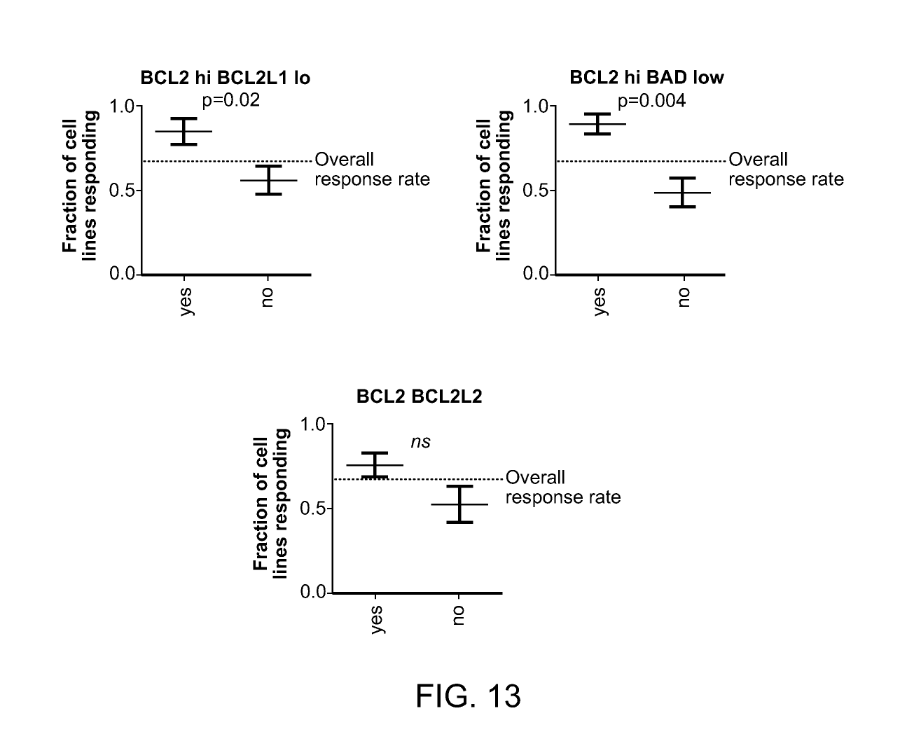

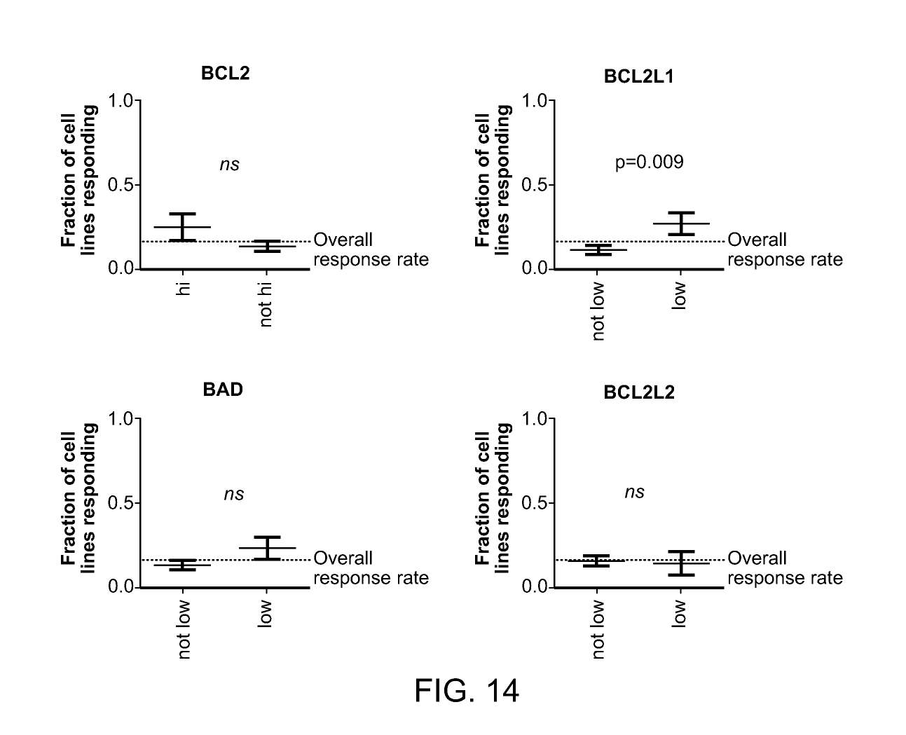

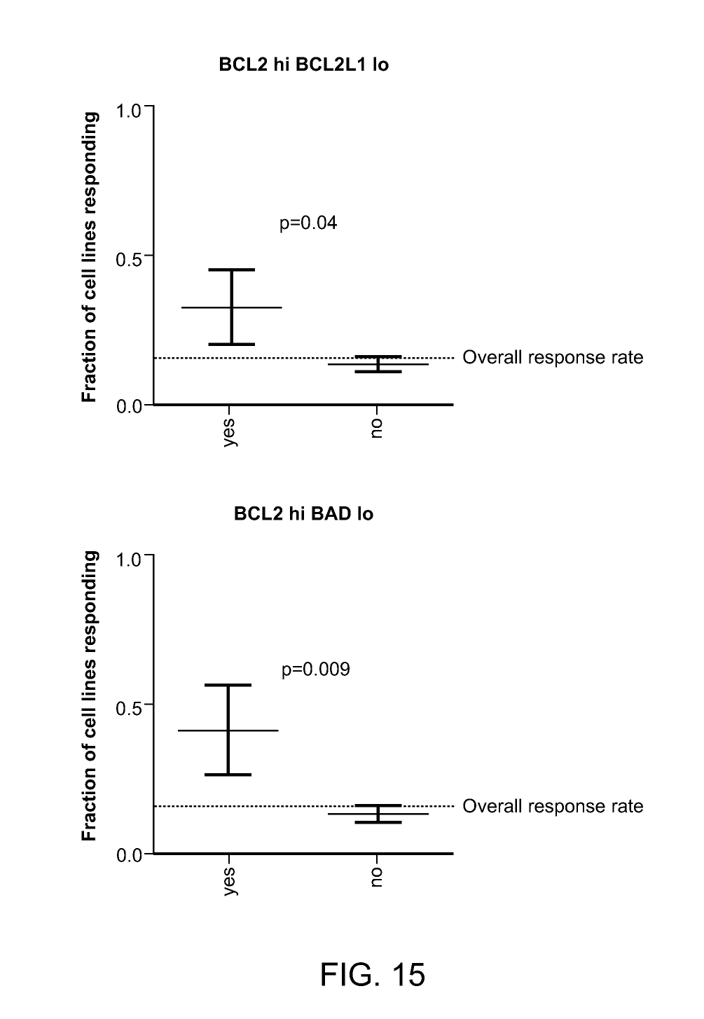

Based on the correlations shown in FIGS. 8 and 9, cell lines expressing high levels of BCL2 were defined as those expressing an RMA value of BCL2 within the top 33% of all 245 cell lines. Similarly, cell lines expressing low levels of BCL2L1, BCL2L2, and BAD were defined as those expressing RMA values of these genes within the bottom 33% of all cell lines. The fraction of cell lines within these categories responding to the BET inhibitor (9) with a GI50 value equal to or less than 0.25 μM was then determined. As shown in FIG. 10, each of these criteria was able to significantly enrich the fraction of cell lines responding above the overall response rate of 28%. Further enrichment was obtained by combining criteria, with the highest enrichment obtained by selecting for cell lines with high expression of BCL2 and low expression of BCL2L1 (65%±7% response rate) or high expression of BCL2 and low expression of BAD (75%±6% response rate) (FIG. 11). Similar enrichment of responsive cell lines is obtained when cell lines of hematologic origin are considered separately from those of solid tumor origin (FIG. 12-15). As shown in FIG. 17, expression levels of BCL2 and BCL2L1 as determined by q-RTPCR are able to predict cell line response to BET inhibition to a comparable extent to values determined by microarray analysis, suggesting that this method of patient stratification may be applicable to more clinically relevant methods of measuring gene expression.

### Selection by Expression of BCL2, BCL2L1, and BAD Enriches for Highly Responding Cancer Subtypes

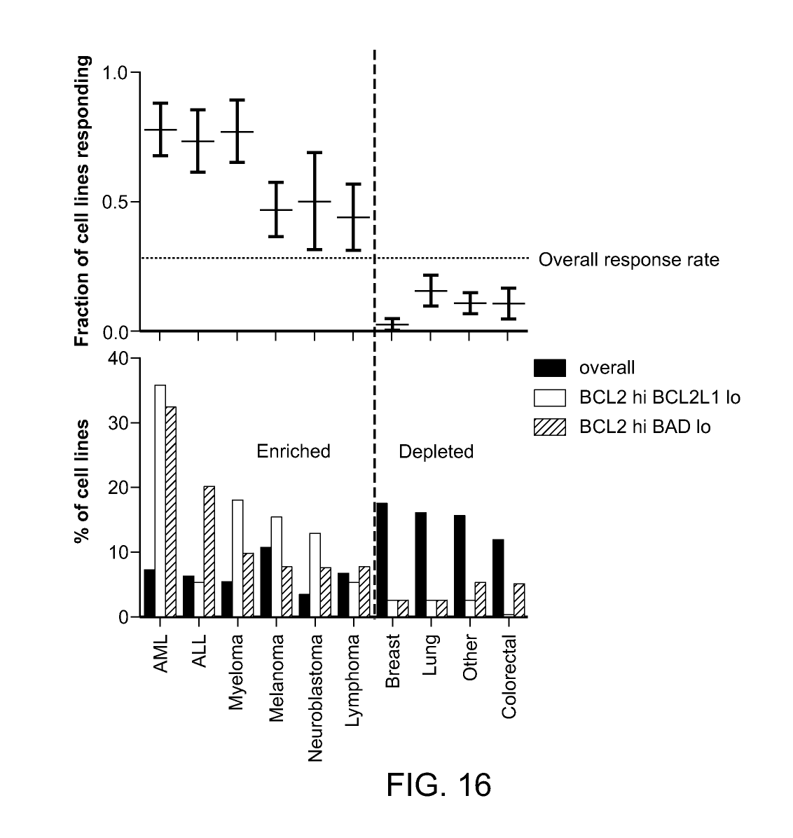

The enrichment of specific subtypes can be observed when cell lines are selected based on BCL2, BCL2L1, and BAD expression. As shown in FIG. 16, cell lines of acute myeloid leukemia (AML), multiple myeloma, melanoma, and neuroblastoma are enriched following selection for cell lines expression high levels of BCL2 and low levels of BCL2L1. Cell lines of AML, multiple myeloma, neuroblastoma, lymphoma, and acute lymphoid leukemia (ALL) origin are enriched following selection for BCL2 high and BAD low. In contrast, following both selection criteria, cell lines of lung cancer, breast cancer, colorectal carcinoma, and other tissues of origin are depleted. The degree of enrichment of a given cancer subtype is correlated with the fraction of cell lines of that subtype that respond to BET inhibitor treatment with a G150 of less than 0.25 μM.

While we have described a number of embodiments of this, it is apparent that our basic examples may be altered to provide other embodiments that utilize the compounds and methods of this disclosure. Therefore, it will be appreciated that the scope of this disclosure is to be defined by the appended claims rather than by the specific embodiments that have been represented by way of example.

The contents of all references (including literature references, issued patents, published patent applications, and co-pending patent applications) cited throughout this application are hereby expressly incorporated herein in their entireties by reference. Unless otherwise defined, all technical and scientific terms used herein are accorded the meaning commonly known to one with ordinary skill in the art.

1. A method of treating a subject with a cancer which is not overexpressing BCL-xL, comprising administering to the subject an effective amount of a bromodomain inhibitor having the formula: 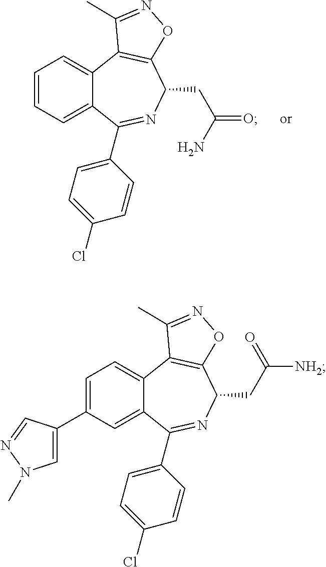 [SMILES: Cc1noc2c1-c1ccccc1C(c1ccc(Cl)cc1)=N[C@H]2CC(N)=O ; Cc1noc2c1-c1ccc(-c3cnn(C)c3)cc1C(c1ccc(Cl)cc1)=N[C@H]2CC(N)=O]
or pharmaceutically acceptable salt thereof.

2. A method of treating a subject with a cancer which is not overexpressing BCL-w, comprising administering to the subject an effective amount of a bromodomain inhibitor having the formula: 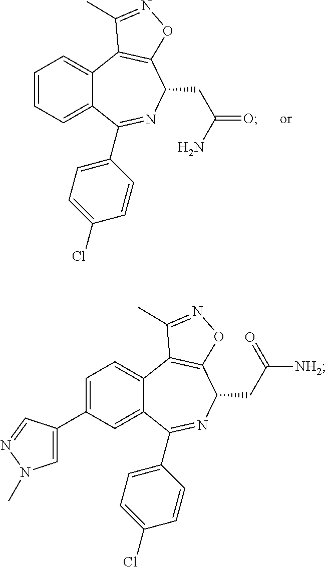 [SMILES: Cc1noc2c1-c1ccccc1C(c1ccc(Cl)cc1)=N[C@H]2CC(N)=O ; Cc1noc2c1-c1ccc(-c3cnn(C)c3)cc1C(c1ccc(Cl)cc1)=N[C@H]2CC(N)=O]
or pharmaceutically acceptable salt thereof.

3. A method of treating a subject with a cancer which is not overexpressing BAD, comprising administering to the subject an effective amount of a bromodomain inhibitor having the formula: 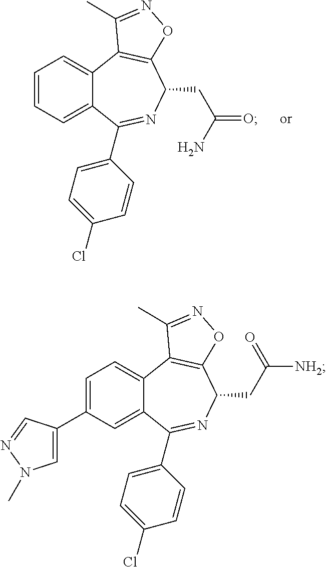 [SMILES: Cc1noc2c1-c1ccccc1C(c1ccc(Cl)cc1)=N[C@H]2CC(N)=O ; Cc1noc2c1-c1ccc(-c3cnn(C)c3)cc1C(c1ccc(Cl)cc1)=N[C@H]2CC(N)=O]
or pharmaceutically acceptable salt thereof.

4. A method of treating a subject with a cancer which is overexpressing BCL-2, comprising administering to the subject an effective amount of a bromodomain inhibitor having the formula: 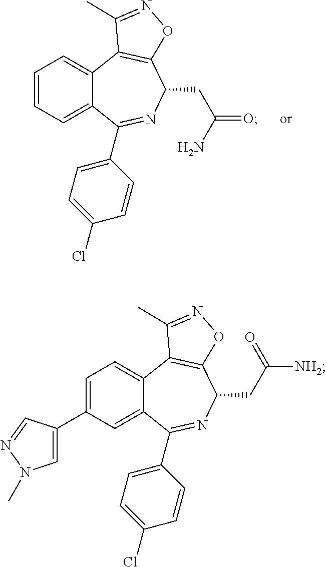 [SMILES: Cc1noc2c1-c1ccccc1C(c1ccc(Cl)cc1)=N[C@H]2CC(N)=O ; Cc1noc2c1-c1ccc(-c3cnn(C)c3)cc1C(c1ccc(Cl)cc1)=N[C@H]2CC(N)=O]
or pharmaceutically acceptable salt thereof.

5. The method of claim 1, wherein prior to treatment, the cancer was determined to not be overexpressing BCL-xL.

6-26. (canceled)

27. The method of claim 1, wherein overexpressing comprises a level of expression of mRNA or protein in a sample of a tumor that is 1.5 fold above the level in a tissue-matched non-malignant cell from the same patient; or comprises greater than 2 copies of the genes for BCL-2 in a tumor sample; or comprises a tumor sample containing genomic translocations involving these genes that are known to promote high expression; or comprises a tumor sample in which >10% of cells express BCL-2 as determined by immunohistochemistry techniques; or comprises an amount of cellular BCL-2 above 0.1 ng per ug of total cellular protein.

28-30. (canceled)

31. The method of claim 1, wherein the cancer is selected from a midline carcinoma, a neuroblastoma, cancer of the lung (large and small), breast, prostate, thyroid, tongue, mouth, pharynx, esophagus, stomach, intestine, colon, rectum, anal canal, liver, bile duct, pancreas, larynx, bone, joints, soft tissue, skin, uterine, ovary, vulva, vagina, testis, bladder, kidney, ureter, eye, and brain.

32. The method of claim 1, wherein the cancer is a hematological malignancy.

33. (canceled)

34. The method of claim 2, wherein prior to treatment, the cancer was determined to not be overexpressing BCL-w.

35. The method of claim 3, wherein prior to treatment, the cancer was determined to not be overexpressing BAD.

36. The method of claim 4, wherein prior to treatment, the cancer was determined to not be overexpressing BCL-2.

37. The method of claim 2, wherein overexpressing comprises a level of expression of mRNA or protein in a sample of a tumor that is 1.5 fold above the level in a tissue-matched non-malignant cell from the same patient; or comprises greater than 2 copies of the genes for BCL-xL in a tumor sample; or comprises a tumor sample containing genomic translocations involving these genes that are known to promote high expression; or comprises a tumor sample in which >10% of cells express BCL-xLas determined by immunohistochemistry techniques; or comprises an amount of cellular BCL-xL above 0.1 ng per ug of total cellular protein.

38. The method of claim 3, wherein overexpressing comprises a level of expression of mRNA or protein in a sample of a tumor that is 1.5 fold above the level in a tissue-matched non-malignant cell from the same patient; or comprises greater than 2 copies of the genes for BCL-w in a tumor sample; or comprises a tumor sample containing genomic translocations involving these genes that are known to promote high expression; or comprises a tumor sample in which >10% of cells express BCL-w as determined by immunohistochemistry techniques; or comprises an amount of cellular BCL-w above 0.1 ng per ug of total cellular protein.

39. The method of claim 4, wherein overexpressing comprises a level of expression of mRNA or protein in a sample of a tumor that is 1.5 fold above the level in a tissue-matched non-malignant cell from the same patient; or comprises greater than 2 copies of the genes for BAD in a tumor sample; or comprises a tumor sample containing genomic translocations involving these genes that are known to promote high expression; or comprises a tumor sample in which >10% of cells express BAD as determined by immunohistochemistry techniques; or comprises an amount of cellular BAD above 0.1 ng per ug of total cellular protein.

40. The method of claim 2, wherein the cancer is selected from a midline carcinoma, a neuroblastoma, cancer of the lung (large and small), breast, prostate, thyroid, tongue, mouth, pharynx, esophagus, stomach, intestine, colon, rectum, anal canal, liver, bile duct, pancreas, larynx, bone, joints, soft tissue, skin, uterine, ovary, vulva, vagina, testis, bladder, kidney, ureter, eye, and brain.

41. The method of claim 3, wherein the cancer is selected from a midline carcinoma, a neuroblastoma, cancer of the lung (large and small), breast, prostate, thyroid, tongue, mouth, pharynx, esophagus, stomach, intestine, colon, rectum, anal canal, liver, bile duct, pancreas, larynx, bone, joints, soft tissue, skin, uterine, ovary, vulva, vagina, testis, bladder, kidney, ureter, eye, and brain.

42. The method of claim 4, wherein the cancer is selected from a midline carcinoma, a neuroblastoma, cancer of the lung (large and small), breast, prostate, thyroid, tongue, mouth, pharynx, esophagus, stomach, intestine, colon, rectum, anal canal, liver, bile duct, pancreas, larynx, bone, joints, soft tissue, skin, uterine, ovary, vulva, vagina, testis, bladder, kidney, ureter, eye, and brain.

43. The method of claim 2, wherein the cancer is a hematological malignancy.

44. The method of claim 3, wherein the cancer is a hematological malignancy.

45. The method of claim 4, wherein the cancer is a hematological malignancy.
